(19) United States
(12) Patent Application Publication
(10) Pub. No.: US 2018/0339015 A1
(43) Pub. Date: Nov. 29, 2018

(54) Therapeutic Nanoparticles Comprising An Antibiotic and Methods of Making and Using Same

(71) Applicant: Pfizer Inc., New York, NY (US)

(72) Inventor: Stephen E. Zale, Hopkinton, MA (US)

(73) Assignee: Pfizer Inc., New York, NY (US)

(21) Appl. No.: 15/778,543

(22) PCT Filed: Nov. 21, 2016
(86) PCT No.: PCT/IB2016/057003

§ 371 Date: May 23, 2018

Related U.S. Application Data
(60) Provisional application No. 62/286,734, filed on Jan. 25, 2016.
(60) Provisional application No. 62/260,075, filed on Nov. 25, 2015.

(51) Int. Cl. A61K 38/12; A61K 47/69; A61P 31/04

(52) U.S. Cl. CPC A61K 38/12; A61P 31/04; A61K 47/6937

(57) ABSTRACT

The present disclosure generally relates to nanoparticles having about 0.2 to about 35 weight percent of a antibiotic therapeutic agent; about 0.05 to about 30 weight percent of a hydrophobic acid; and about 35 to about 99.75 weight percent of biocompatible polymer such as a diblock poly(lactic) acid-poly(ethylene)glycol or diblock poly(lactic)-co-poly(glycolic) acid-poly(ethylene)glycol. Other aspects include methods of making such nanoparticles.

### BACKGROUND

Systems that deliver certain drugs to a patient (e.g., targeted to a particular tissue or cell type or targeted to a specific diseased tissue but not normal tissue) or that control release of drugs have long been recognized as beneficial.

For example, therapeutics that include an active drug and that are, e.g., targeted to a particular tissue or cell type or targeted to a specific diseased tissue but not to normal tissue, may reduce the amount of the drug in tissues of the body that are not targeted. This is particularly important when treating a condition such as cancer where it is desirable that a cytotoxic dose of the drug is delivered to cancer cells without killing the surrounding non-cancerous tissue. Effective drug targeting may reduce the undesirable and sometimes life threatening side effects common in anticancer therapy. In addition, such therapeutics may allow drugs to reach certain tissues they would otherwise be unable to reach.

Therapeutics that offer controlled release and/or targeted therapy also must be able to deliver an effective amount of drug, which is a known limitation in other nanoparticle delivery systems. For example, it can be a challenge to prepare nanoparticle systems that have an appropriate amount of drug associated with each nanoparticle, while keeping the size of the nanoparticles small enough to have advantageous delivery properties.

Antibiotics represent an important group of therapeutic agents. However, some classes of antibiotics, such as the polymyxin/colistin class, are highly hydrophilic and will not partition into an oil phase at any pH. In the face of diminishing therapeutic options for the treatment of infections caused by multidrug-resistant Gram-negative bacteria, clinicians are increasingly using colistin. Due to undesired toxicity issues, primarily nephrotoxicity, after i.v. administration of colistin sulphate (CS), colistin is generally administered as a prodrug, colistin methanesulfonate (CMS). CMS is associated with variable pharmacokinetics (PK) and poor deposition into diseased tissues, and inefficient conversion of the prodrug to the active moiety.

Accordingly, a need exists for therapeutic nanoparticles, and methods of making such nanoparticles, that are capable of delivering therapeutic levels of antibiotics such as the polymyxin/colistin class of antibiotics to treat diseases and infections, while also reducing patient side effects.

### SUMMARY

Described herein are polymeric nanoparticles that include an antibiotic, e.g. a polymyxin/colistin antibiotic, and methods of making and using such therapeutic nanoparticles.

For example, disclosed herein is a therapeutic nanoparticle comprising about 0.2 to about 35 weight percent of an antibiotic (e.g., colistin) therapeutic agent; about 0.05 to about 30 weight percent of a hydrophobic acid; and about 35 to about 99.75 weight percent of a diblock poly(lactic) acid-poly(ethylene)glycol copolymer or a diblock poly(lactic)-co-poly(glycolic) acid-poly(ethylene)glycol copolymer, wherein the therapeutic nanoparticle comprises about 10 to about 30 weight percent poly(ethylene)glycol. A contemplated therapeutic nanoparticle may have a hydrodynamic diameter of about 60 to about 150 nm, about 90 to about 140 nm, about 90 to about 130 nm, or about 100 to about 125 nm.

Contemplated herein are nanoparticles that include polymyxin/colistin antibiotic therapeutic agents, such as any antibiotic from the polymyxin/colistin class, such as colistin sulfate, colistimethate sodium (colistin methanesulfonate sodium, colistin sulfomethate sodium), and polymyxin A, B, C, D, and E. Polymyxin B includes polymyxin B1 and B2, and polymyxin E includes E1 (colistin A) and E2 (colistin B).

In still another aspect, a pharmaceutically acceptable composition is provided. The pharmaceutically acceptable composition may comprise a plurality of therapeutic nanoparticles as described herein and a pharmaceutically acceptable excipient.

In yet another aspect, a method of treating an infection in a patient in need thereof is provided. The method comprises administering to the patient a therapeutically effective amount of a composition comprising a therapeutic nanoparticle as described herein. In some embodiments, the infection may be caused by a gram negative bacteria. In other embodiments, the infection may be a panresistant nosocomial infections, especially due to Pseudomonas, Acinetobacter, or Klebsiella pneumonia.

### BRIEF DESCRIPTION OF THE DRAWINGS

FIG. 1 depicts a pictorial representation of one embodiment of a disclosed nanoparticle.

FIG. 2 is flow chart for an emulsion process for forming a disclosed nanoparticle.

FIG. 3A is a flow diagram for a disclosed emulsion process.

FIG. 3B is a flow diagram for a disclosed emulsion process.

FIG. 4 depicts in vitro release (IVR) profiles for colistin-containing nanoparticle formulations including decanoic acid (DEC), pamoic acid (PAM), xinaphoic acid (XIN), or deoxycholic acid (DEOC).

FIG. 5 depicts in vitro release (IVR) profiles for colistin-containing nanoparticle formulations A and B.

FIG. 6 depicts in vitro release (IVR) profiles for colistin-containing nanoparticle formulations F, US, I and S.

FIG. 7 depicts in vitro release (IVR) profiles for polymixin B-containing nanoparticle formulations including the indicated acid.

FIG. 8 depicts pharmacokinetics for free colistin sulphate (CS) compared to formulation F and S colistin-containing nanoparticles.

FIG. 9 depicts tolerability of formulation S colistin-containing nanoparticles in CD-1 mice following a single i.v. dose of the indicated size.

FIG. 10 depicts kidney histology after s.c. administration of conventional colistin sulphate (left), s.c. administration of formulation S colistin-containing nanoparticles (middle), or i.v. administration of colistin-containing nanoparticles (right).

FIG. 11 depicts comparable efficacy studies of conventional drug (CS) and formulation S colistin-containing nanoparticles in a K. pneumonia lung infection model FIG. 12 depicts comparable efficacy studies of conventional drug (CS) and CS-NPs of the indicated formulations in a Klebsiella thigh infection model.

FIG. 13 depicts accumulation of CS-NPs in a Klebsiella thigh infection model.

FIG. 14 depicts a comparison of bacterial loads in mouse thighs after 1, 4, and 21 hours treated by IV with either vincristine or exemplary disclosed nanoparticle that includes vincristine; mice were immunocompetent CD-1 females; the bacterium used was K. pneumoniae

FIG. 15 depicts (Left) thigh weight of rats for infected vs. un-infected (Right).

FIG. 16 depicts (Left) plasma levels of vincristine resulting from treatment with vincristine only (indicated by “Vin” axis label) vs. vincristine-containing nanoparticles (indicated by “Vin-Acc” axis label) and (Right) an expansion of the left side of the graph that shows the plasma levels of vincristine-only treated rats.

FIG. 17 depicts thigh tissue levels of vincristine vs. an exemplary disclosed nanoparticle that includes vincristine.

FIG. 18 depicts thigh tissue levels of vincristine for an exemplary disclosed nanoparticle that includes vincristine for left thigh (infected) vs. right thigh (uninfected).

FIG. 19 depicts a graph of vincristine release in vitro from an exemplary disclosed nanoparticle that includes vincristine.

### DETAILED DESCRIPTION

Described herein are polymeric nanoparticles that include an antibiotic therapeutic agent, and methods of making and using such therapeutic nanoparticles. In some embodiments, inclusion (i.e., doping) of a hydrophobic acid (e.g., a fatty acid and/or a bile acid) in a disclosed nanoparticle and/or included in a nanoparticle preparation process may result in nanoparticles that include improved drug loading. Furthermore, in certain embodiments, nanoparticles that include and/or are prepared in the presence of the hydrophobic acid may exhibit improved controlled release properties. For example, disclosed nanoparticles may more slowly release the antibiotic therapeutic agent as compared to nanoparticles prepared in the absence of the hydrophobic acid.

Without wishing to be bound by any theory, it is believed that the disclosed nanoparticle formulations that include a hydrophobic acid (e.g., fatty acid and/or bile acid) have significantly improved formulation properties (e.g., drug loading and/or release profile) through formation of a hydrophobic ion-pair (HIP), between a therapeutic agent and an acid. As used herein, a HIP is a pair of oppositely charged ions held together by Coulombic attraction. Also without wishing to be bound by any theory, in some embodiments, a HIP can be used to increase the hydrophobicity of a therapeutic agent containing ionizable groups (e.g., amines). In some embodiments, a therapeutic agent with increased hydrophobicity can be beneficial for nanoparticle formulations and result in a HIP formation that may provide higher solubility of the therapeutic agent in organic solvents. HIP formation, as contemplated herein, can result in nanoparticles having for example, increased drug loading. Slower release of the therapeutic agent from the nanoparticles may also occur, for example in some embodiments, due to a decrease in the therapeutic agent's solubility in aqueous solution. Furthermore, complexing the therapeutic agent with large hydrophobic counter ions may slow diffusion of the therapeutic agent within the polymeric matrix. Advantageously, HIP formation occurs without the need for covalent conjugation of the hydrophobic group to the therapeutic agent.

Nanoparticles disclosed herein include one, two, three or more biocompatible and/or biodegradable polymers. For example, a contemplated nanoparticle may include about 35 to about 99.75 weight percent, in some embodiments about 50 to about 99.75 weight percent, in some embodiments about 50 to about 99.5 weight percent, in some embodiments about 50 to about 99 weight percent, in some embodiments about 50 to about 98 weight percent, in some embodiments about 50 to about 97 weight percent, in some embodiments about 50 to about 96 weight percent, in some embodiments about 50 to about 95 weight percent, in some embodiments about 50 to about 94 weight percent, in some embodiments about 50 to about 93 weight percent, in some embodiments about 50 to about 92 weight percent, in some embodiments about 50 to about 91 weight percent, in some embodiments about 50 to about 90 weight percent, in some embodiments about 50 to about 85 weight percent, and in some embodiments about 50 to about 80 weight percent of one or more block copolymers that include a biodegradable polymer and poly(ethylene glycol) (PEG), and about 0 to about 50 weight percent of a biodegradable homopolymer.

Contemplated herein are antibiotics such as antibiotics produced by certain strains of Paenibacillus polymyxa or Bacillus polymyxa.

In some embodiments, disclosed nanoparticles may include about 0.2 to about 35 weight percent, about 0.2 to about 20 weight percent, about 0.2 to about 10 weight percent, about 0.2 to about 5 weight percent, about 0.5 to about 5 weight percent, about 0.75 to about 5 weight percent, about 1 to about 5 weight percent, about 2 to about 5 weight percent, about 3 to about 5 weight percent, about 1 to about 20 weight percent, about 2 to about 20 weight percent, about 5 to about 20 weight percent, about 1 to about 15 weight percent, about 2 to about 15 weight percent, about 3 to about 15 weight percent, about 4 to about 15 weight percent, about 5 to about 15 weight percent, about 1 to about 10 weight percent, about 2 to about 10 weight percent, about 3 to about 10 weight percent, about 4 to about 10 weight percent, about 5 to about 10 weight percent, about 10 to about 30 weight percent, or about 15 to about 25 weight percent of an antibiotic therapeutic agent.

In certain embodiments, disclosed nanoparticles comprise a hydrophobic acid (e.g., a fatty acid and/or bile acid) and/or are prepared by a process that includes a hydrophobic acid. Such nanoparticles may have a higher drug loading than nanoparticles prepared by a process without a hydrophobic acid. For example, drug loading (e.g., by weight) of disclosed nanoparticles prepared by a process comprising the hydrophobic acid may be between about 2 times to about 10 times higher, or even more, than disclosed nanoparticles prepared by a process without the hydrophobic acid. In some embodiments, the drug loading (by weight) of disclosed nanoparticles prepared by a first process comprising the hydrophobic acid may be at least about 2 times higher, at least about 3 times higher, at least about 4 times higher, at least about 5 times higher, or at least about 10 times higher than disclosed nanoparticles prepared by a second process, where the second process is identical to the first process except that the second process does not include the hydrophobic acid.

Any suitable hydrophobic acid is contemplated. In some embodiments, the hydrophobic acid may be a carboxylic acid (e.g., a monocarboxylic acid, dicarboxylic acid, tricarboxylic acid, or the like), a sulfinic acid, a sulfenic acid, or a sulfonic acid. In some cases, a contemplated hydrophobic acid may include a mixture of two or more acids. In some cases, a salt of a hydrophobic acid may be used in a formulation.

For example, a disclosed carboxylic acid may be an aliphatic carboxylic acid (e.g., a carboxylic acid having a cyclic or acyclic, branched or unbranched, hydrocarbon chain). Disclosed carboxylic acids may, in some embodiments, be substituted with one or more functional groups including, but not limited to, halogen (i.e., F, Cl, Br, and I), sulfonyl, nitro, and oxo. In certain embodiments, a disclosed carboxylic acid may be unsubstituted.

Exemplary carboxylic acids may include a substituted or unsubstituted fatty acid (e.g., C₆-C₅₀ fatty acid). In some instances, the fatty acid may be a C₁₀-C₂₀ fatty acid. In other instances, the fatty acid may be a C₁₅-C₂₀ fatty acid. The fatty acid may, in some cases, be saturated. In other embodiments, the fatty acid may be unsaturated. For instance, the fatty acid may be a monounsaturated fatty acid or a polyunsaturated fatty acid. In some embodiments, a double bond of an unsaturated fatty acid group can be in the cis conformation. In some embodiments, a double bond of an unsaturated fatty acid can be in the trans conformation. Unsaturated fatty acids include, but are not limited to, omega-3, omega-6, and omega-9 fatty acids.

Non-limiting examples of saturated fatty acids include caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, heneicosanoic acid, behenic acid, tricosanoic acid, lignoceric acid, pentacosanoic acid, cerotic acid, heptacosanoic acid, montanic acid, nonacosanoic acid, melissic acid, henatriacontanoic acid, lacceroic acid, psyllic acid, geddic acid, ceroplastic acid, hexatriacontanoic acid, and combinations thereof.

Non-limiting examples of unsaturated fatty acids include hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, tetracosahexaenoic acid, linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid, oleic acid, eicosenoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, podocarpic acid, palmitoleic acid, vaccenic acid, gadoleic acid, erucic acid, and combinations thereof.

Other non-limiting examples of hydrophobic acids include aromatic acids, such as 1-hydroxy-2-naphthoic acid, naphthalene-1, 5-disulfonic acid, naphthalene-2-sulfonic acid, pamoic acid, cinnamic acid, phenylacetic acid, and combinations thereof.

In some embodiments, the hydrophobic acid may be a bile acid. Non-limiting examples of bile acids include chenodeoxycholic acid, ursodeoxycholic acid, deoxycholic acid, hycholic acid, beta-muricholic acid, cholic acid, an amino acid-conjugated bile acid, and combinations thereof. An amino-acid conjugated bile acid may be conjugated to any suitable amino acid. In some embodiments, the amino acid-conjugated bile acid is a glycine-conjugated bile acid or a taurine-conjugated bile acid.

In some instances, a contemplated acid may have a molecular weight of less than about 1000 Da, in some embodiments less than about 500 Da, in some embodiments less than about 400 Da, in some embodiments less than about 300 Da, in some embodiments less than about 250 Da, in some embodiments less than about 200 Da, and in some embodiments less than about 150 Da. In some cases, the acid may have a molecular weight of between about 100 Da and about 1000 Da, in some embodiments between about 200 Da and about 800 Da, in some embodiments between about 200 Da and about 600 Da, in some embodiments between about 100 Da and about 300 Da, in some embodiments between about 200 Da and about 400 Da, and in some embodiments between about 300 Da and about 500 Da.

In some embodiments, a hydrophobic acid may be chosen, at least in part, on the basis of the strength of the acid. For example, the hydrophobic acid may have an acid dissociation constant in water (pKₐ) of about −5 to about 7, in some embodiments about −3 to about 5, in some embodiments about −3 to about 4, in some embodiments about −3 to about 3.5, in some embodiments about −3 to about 3, in some embodiments about −3 to about 2, in some embodiments about −3 to about 1, in some embodiments about −3 to about 0.5, in some embodiments about −0.5 to about 0.5, in some embodiments about 1 to about 7, in some embodiments about 2 to about 7, in some embodiments about 3 to about 7, in some embodiments about 4 to about 6, in some embodiments about 4 to about 5.5, in some embodiments about 4 to about 5, and in some embodiments about 4.5 to about 5, determined at 25° C. In some embodiments, the acid may have a pKₐ of less than about 7, less than about 5, less than about 3.5, less than about 3, less than about 2, less than about 1, or less than about 0, determined at 25° C.

In some embodiments, a contemplated hydrophobic acid may have a phase transition temperature that is advantageous, for example, for improving the properties of the therapeutic nanoparticles in the final therapeutic nanoparticles. For instance, the acid may have a melting point of less than about 300° C., in some cases less than about 100° C., and in some cases less than about 50° C. In certain embodiments, the acid may have a melting point of between about 5° C. and about 25° C., in some cases between about 15° C. and about 50° C., in some cases between about 30° C. and about 100° C., in some cases between about 75° C. and about 150° C., in some cases between about 125° C. and about 200° C., in some cases between about 150° C. and about 250° C., and in some cases between about 200° C. and about 300° C. In some cases, the acid may have a melting point of less than about 15° C., in some cases less than about 10° C., or in some cases less than about 0° C. In certain embodiments, the acid may have a melting point of between about −30° C. and about 0° C. or in some cases between about −20° C. and about −10° C.

For example, an acid for use in methods and nanoparticles disclosed herein may be chosen, at least in part, on the basis of the solubility of the antibiotic therapeutic agent in a solvent comprising the acid. For example, in some embodiments, a polymyxin/colistin antibiotic therapeutic agent dissolved in a solvent comprising the acid may have a solubility of between about 700 mg/mL to about 900 mg/mL, between about 600 mg/mL to about 800 mg/mL, between about 500 mg/mL to about 700 mg/mL to about 800 mg/mL, between about 15 mg/mL to about 200 mg/mL, between about 20 mg/mL to about 200 mg/mL, between about 25 mg/mL to about 200 mg/mL, between about 50 mg/mL to about 200 mg/mL, between about 75 mg/mL to about 200 mg/mL, between about 100 mg/mL to about 200 mg/mL, between about 125 mg/mL to about 175 mg/mL, between about 15 mg/mL to about 50 mg/mL, between about 25 mg/mL to about 75 mg/mL. In some embodiments, a antibiotic therapeutic agent dissolved in a solvent comprising the acid may have a solubility greater than about 10 mg/mL, greater than about 50 mg/mL, or greater than about 100 mg/mL. In some embodiments, a antibiotic therapeutic agent dissolved in a solvent comprising the hydrophobic acid (e.g., a first solution consisting of the therapeutic agent, solvent, and hydrophobic acid) may have a solubility of at least about 2 times greater, in some embodiments at least about 5 times greater, in some embodiments at least about 10 times greater, in some embodiments at least about 20 times greater, in some embodiments about 2 times to about 20 times greater or in some embodiments about 10 times to about 20 times greater than when the antibiotic therapeutic agent is dissolved in a solvent that does not contain the hydrophobic acid (e.g., a second solution consisting of the therapeutic agent and the solvent).

In some instances, the concentration of acid in a drug solution (i.e., a antibiotic therapeutic agent solution) may be between about 1 weight percent and about 30 weight percent, in some embodiments between about 2 weight percent and about 30 weight percent, in some embodiments between about 3 weight percent and about 30 weight percent, in some embodiments between about 4 weight percent and about 30 weight percent, in some embodiments between about 5 weight percent and about 30 weight percent, in some embodiments between about 6 weight percent and about 30 weight percent, in some embodiments between about 8 weight percent and about 30 weight percent, in some embodiments between about 10 weight percent and about 30 weight percent, in some embodiments between about 12 weight percent and about 30 weight percent, in some embodiments between about 14 weight percent and about 30 weight percent, in some embodiments between about 16 weight percent and about 30 weight percent, in some embodiments between about 1 weight percent and about 5 weight percent, in some embodiments between about 3 weight percent and about 9 weight percent, in some embodiments between about 6 weight percent and about 12 weight percent, in some embodiments between about 9 weight percent and about 15 weight percent, in some embodiments between about 12 weight percent and about 18 weight percent, and in some embodiments between about 15 weight percent and about 21 weight percent. In certain embodiments, the concentration of hydrophobic acid in a drug solution may be at least about 1 weight percent, in some embodiments at least about 2 weight percent, in some embodiments at least about 3 weight percent, in some embodiments at least about 5 weight percent, in some embodiments at least about 10 weight percent, in some embodiments at least about 15 weight percent, and in some embodiments at least about 20 weight percent.

In certain embodiments, the hydrophobic acid may have a solubility of less than about 2 g per 100 mL of water, in some embodiments less than about 1 g per 100 mL of water, in some embodiments less than about 100 mg per 100 mL of water, in some embodiments less than about 10 mg per 100 mL of water, and in some embodiments less than about 1 mg per 100 mL of water, determined at 25° C. In other embodiments, the acid may have a solubility of between about 1 mg per 100 mL of water to about 2 g per 100 mL of water, in some embodiments between about 1 mg per 100 mL of water to about 1 g per 100 mL of water, in some embodiments between about 1 mg per 100 mL of water to about 500 mg per 100 mL of water, and in some embodiments between about 1 mg per 100 mL of water to about 100 mg per 100 mL of water, determined at 25° C. In some embodiments, the hydrophobic acid may be essentially insoluble in water at 25° C.

In some embodiments, disclosed nanoparticles may be essentially free of the hydrophobic acid used during the preparation of the nanoparticles. In other embodiments, disclosed nanoparticles may comprise the hydrophobic acid. For instance, in some embodiments, the acid content in disclosed nanoparticles may be between about 0.05 weight percent to about 30 weight percent, in some embodiments between about 0.5 weight percent to about 30 weight percent, in some embodiments between about 1 weight percent to about 30 weight percent, in some embodiments between about 2 weight percent to about 30 weight percent, in some embodiments between about 3 weight percent to about 30 weight percent, in some embodiments between about 5 weight percent to about 30 weight percent, in some embodiments between about 7 weight percent to about 30 weight percent, in some embodiments between about 10 weight percent to about 30 weight percent, in some embodiments between about 15 weight percent to about 30 weight percent, in some embodiments between about 20 weight percent to about 30 weight percent, in some embodiments between about 0.05 weight percent to about 0.5 weight percent, in some embodiments between about 0.05 weight percent to about 5 weight percent, in some embodiments between about 1 weight percent to about 5 weight percent, in some embodiments between about 3 weight percent to about 10 weight percent, in some embodiments between about 5 weight percent to about 15 weight percent, and in some embodiments between about 10 weight percent to about 20 weight percent.

In some embodiments, disclosed nanoparticles substantially immediately release (e.g., over about 1 minute to about 30 minutes, about 1 minute to about 25 minutes, about 5 minutes to about 30 minutes, about 5 minutes to about 1 hour, about 1 hour, or about 24 hours) less than about 2%, less than about 5%, less than about 10%, less than about 15%, less than about 20%, less than about 25%, less than about 30%, or less than 40% of the antibiotic therapeutic agent, for example when placed in a phosphate buffer solution at room temperature (e.g., 25° C.) and/or at 37° C. In certain embodiments, nanoparticles comprising a antibiotic therapeutic agent may release the antibiotic therapeutic agent when placed in an aqueous solution (e.g., a phosphate buffer solution), e.g., at 25° C. and/or at 37° C., at a rate substantially corresponding to about 0.01 to about 50%, in some embodiments about 0.01 to about 25%, in some embodiments about 0.01 to about 15%, in some embodiments about 0.01 to about 10%, in some embodiments about 1 to about 40%, in some embodiments about 5 to about 40%, and in some embodiments about 10 to about 40% of the antibiotic therapeutic agent released over about 1 hour. In some embodiments, nanoparticles comprising a antibiotic therapeutic agent may release the antibiotic therapeutic agent when placed in an aqueous solution (e.g., a phosphate buffer solution), e.g., at 25° C. and/or at 37° C., at a rate substantially corresponding to about 10 to about 70%, in some embodiments about 10 to about 45%, in some embodiments about 10 to about 35%, or in some embodiments about 10 to about 25%, of the polymyxin/colistin antibiotic therapeutic agent released over about 4 hours.

In some embodiments, disclosed nanoparticles may substantially retain the polymyxin/colistin antibiotic therapeutic agent, e.g., for at least about 1 minute, at least about 1 hour, or more, when placed in a phosphate buffer solution at 37° C.

In one embodiment, disclosed therapeutic nanoparticles may include a targeting ligand, e.g., a low-molecular weight PSMA ligand effective for targeting or binding to prostate specific membrane antigen. In certain embodiments, the low-molecular weight ligand is conjugated to a polymer, and the nanoparticle comprises a certain ratio of ligand-conjugated polymer (e.g., PLA-PEG-Ligand) to non-functionalized polymer (e.g., PLA-PEG or PLGA-PEG). The nanoparticle can have an optimized ratio of these two polymers such that an effective amount of ligand is associated with the nanoparticle for treatment of a disease or disorder, such as cancer. For example, an increased ligand density may increase target binding (cell binding/target uptake), making the nanoparticle “target specific.” Alternatively, a certain concentration of non-functionalized polymer (e.g., non-functionalized PLGA-PEG copolymer) in the nanoparticle can control inflammation and/or immunogenicity (i.e., the ability to provoke an immune response), and allow the nanoparticle to have a circulation half-life that is adequate for the treatment of a disease or disorder (e.g., prostate cancer). Furthermore, the non-functionalized polymer may, in some embodiments, lower the rate of clearance from the circulatory system via the reticuloendothelial system (RES). Thus, the non-functionalized polymer may provide the nanoparticle with characteristics that may allow the particle to travel through the body upon administration. In some embodiments, a non-functionalized polymer may balance an otherwise high concentration of ligands, which can otherwise accelerate clearance by the subject, resulting in less delivery to the target cells.

In some embodiments, nanoparticles disclosed herein may include functionalized polymers conjugated to a ligand that constitute approximately 0.1-50, e.g., 0.1-30, e.g., 0.1-20, e.g., 0.1-10 mole percent of the entire polymer composition of the nanoparticle (i.e., functionalized+non-functionalized polymer). Also disclosed herein, in another embodiment, are nanoparticles that include a polymer conjugated (e.g., covalently with (i.e., through a linker (e.g., an alkylene linker)) or a bond) with one or more low-molecular weight ligands, wherein the weight percent low-molecular weight ligand with respect to total polymer is between about 0.001 and 5, e.g., between about 0.001 and 2, e.g., between about 0.001 and 1.

In some embodiments, disclosed nanoparticles may be able to bind efficiently to or otherwise associate with a biological entity, for example, a particular membrane component or cell surface receptor. Targeting of a therapeutic agent (e.g., to a particular tissue or cell type, to a specific diseased tissue but not to normal tissue, etc.) is desirable for the treatment of tissue specific diseases such as solid tumor cancers (e.g., prostate cancer). For example, in contrast to systemic delivery of a cytotoxic anti-cancer agent, the nanoparticles disclosed herein may substantially prevent the agent from killing healthy cells. Additionally, disclosed nanoparticles may allow for the administration of a lower dose of the agent (as compared to an effective amount of agent administered without disclosed nanoparticles or formulations) which may reduce the undesirable side effects commonly associated with traditional chemotherapy.

In general, a “nanoparticle” refers to any particle having a diameter of less than 1000 nm, e.g., about 10 nm to about 200 nm. Disclosed therapeutic nanoparticles may include nanoparticles having a diameter of about 60 to about 120 nm, or about 70 to about 120 nm, or about 80 to about 120 nm, or about 90 to about 120 nm, or about 100 to about 120 nm, or about 60 to about 130 nm, or about 70 to about 130 nm, or about 80 to about 130 nm, or about 90 to about 130 nm, or about 100 to about 130 nm, or about 110 to about 130 nm, or about 60 to about 140 nm, or about 70 to about 140 nm, or about 80 to about 140 nm, or about 90 to about 140 nm, or about 100 to about 140 nm, or about 110 to about 140 nm, or about 60 to about 150 nm, or about 70 to about 150 nm, or about 80 to about 150 nm, or about 90 to about 150 nm, or about 100 to about 150 nm, or about 110 to about 150 nm, or about 120 to about 150 nm.

### Polymers

In some embodiments, the nanoparticles may comprise a matrix of polymers and a therapeutic agent. In some embodiments, a therapeutic agent and/or targeting moiety (i.e., a low-molecular weight PSMA ligand) can be associated with at least part of the polymeric matrix. For example, in some embodiments, a targeting moiety (e.g., ligand) can be covalently associated with the surface of a polymeric matrix. In some embodiments, covalent association is mediated by a linker. The therapeutic agent can be associated with the surface of, encapsulated within, surrounded by, and/or dispersed throughout the polymeric matrix.

A wide variety of polymers and methods for forming particles therefrom are known in the art of drug delivery. In some embodiments, the disclosure is directed toward nanoparticles with at least two macromolecules, wherein the first macromolecule comprises a first polymer bound to a low-molecular weight ligand (e.g., targeting moiety); and the second macromolecule comprising a second polymer that is not bound to a targeting moiety. The nanoparticle can optionally include one or more additional, unfunctionalized, polymers.

Any suitable polymer can be used in the disclosed nanoparticles. Polymers can be natural or unnatural (synthetic) polymers. Polymers can be homopolymers or copolymers comprising two or more monomers. In terms of sequence, copolymers can be random, block, or comprise a combination of random and block sequences. Typically, polymers are organic polymers.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments include polydioxanone (PDO), polyhydroxyalkanoate, polyhydroxybutyrate, poly(glycerol sebacate), polyglycolide (i.e., poly(glycolic) acid) (PGA), polylactide (i.e., poly(lactic) acid) (PLA), poly(lactic) acid-co-poly(glycolic) acid (PLGA), polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and ether bonds (e.g., R—O—R′ bonds). In some embodiments, a biodegradable polymer, such as a hydrolyzable polymer, containing carboxylic acid groups, may be conjugated with poly(ethylene glycol) repeat units to form a poly(ester-ether). A polymer (e.g., copolymer, e.g., block copolymer) containing poly(ethylene glycol) repeat units can also be referred to as a “PEGylated” polymer.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA, PEGylated PGA, PEGylated PLGA, and derivatives thereof). In some embodiments, polyesters include, for example, polyanhydrides, poly(ortho ester) PEGylated poly(ortho ester), poly(caprolactone), PEGylated poly(caprolactone), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D, L-lactic acid. The degradation rate of PLGA can be adjusted by altering the lactic acid-glycolic acid ratio. In some embodiments, PLGA can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g., DNA, RNA, or derivatives thereof). Amine-containing polymers such as poly(lysine), polyethylene imine (PEI), and poly(amidoamine) dendrimers are contemplated for use, in some embodiments, in a disclosed particle.

In some embodiments, polymers can be degradable polyesters bearing cationic side chains. Examples of these polyesters include poly(L-lactide-co-L-lysine), poly(serine ester), poly(4-hydroxy-L-proline ester).

It is contemplated that PEG may be terminated and include an end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

Those of ordinary skill in the art will know of methods and techniques for PEGylating a polymer, for example, by using EDC (l-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

In one embodiment, the molecular weight (or e.g., the ratio of molecular weights of, e.g., different blocks of a copolymer) of the polymers can be optimized for effective treatment as disclosed herein. For example, the molecular weight of a polymer may influence particle degradation rate (such as when the molecular weight of a biodegradable polymer can be adjusted), solubility, water uptake, and drug release kinetics. For example, the molecular weight of the polymer (or e.g., the ratio of molecular weights of, e.g., different blocks of a copolymer) can be adjusted such that the particle biodegrades in the subject being treated within a reasonable period of time (ranging from a few hours to 1-2 weeks, 3-4 weeks, 5-6 weeks, 7-8 weeks, etc.).

A disclosed particle can for example comprise a diblock copolymer of PEG and PL(G)A, wherein for example, the PEG portion may have a number average molecular weight of about 1,000-20,000, e.g., about 2,000-20,000, e.g., about 2 to about 10,000, and the PL(G)A portion may have a number average molecular weight of about 5,000 to about 20,000, or about 5,000-100,000, e.g., about 20,000-70,000, e.g., about 15,000-50,000.

For example, disclosed here is an exemplary therapeutic nanoparticle that includes about 10 to about 99 weight percent poly(lactic) acid-poly(ethylene)glycol copolymer or poly(lactic)-co-poly (glycolic) acid-poly(ethylene)glycol copolymer, or about 20 to about 80 weight percent, about 40 to about 80 weight percent, or about 30 to about 50 weight percent, or about 70 to about 90 weight percent poly(lactic) acid-poly(ethylene)glycol copolymer or poly(lactic)-co-poly (glycolic) acid-poly(ethylene)glycol copolymer. Exemplary poly(lactic) acid-poly(ethylene)glycol copolymers can include a number average molecular weight of about 15 to about 20 kDa, or about 10 to about 25 kDa of poly(lactic) acid and a number average molecular weight of about 4 to about 6, or about 2 kDa to about 10 kDa of poly(ethylene)glycol.

In some embodiments, the poly(lactic) acid-poly(ethylene)glycol copolymer may have a poly(lactic) acid number average molecular weight fraction of about 0.6 to about 0.95, in some embodiments between about 0.7 to about 0.9, in some embodiments between about 0.6 to about 0.8, in some embodiments between about 0.7 to about 0.8, in some embodiments between about 0.75 to about 0.85, in some embodiments between about 0.8 to about 0.9, and in some embodiments between about 0.85 to about 0.95. It should be understood that the poly(lactic) acid number average molecular weight fraction may be calculated by dividing the number average molecular weight of the poly(lactic) acid component of the copolymer by the sum of the number average molecular weight of the poly(lactic) acid component and the number average molecular weight of the poly(ethylene)glycol component.

Disclosed nanoparticles may optionally include about 1 to about 50 weight percent poly(lactic) acid or poly(lactic) acid-co-poly (glycolic) acid (which does not include PEG), or may optionally include about 1 to about 50 weight percent, or about 10 to about 50 weight percent or about 30 to about 50 weight percent poly(lactic) acid or poly(lactic) acid-co-poly (glycolic) acid. For example, poly(lactic) or poly(lactic)-co-poly(glycolic) acid may have a number average molecule weight of about 5 to about 15 kDa, or about 5 to about 12 kDa. Exemplary PLA may have a number average molecular weight of about 5 to about 10 kDa. Exemplary PLGA may have a number average molecular weight of about 8 to about 12 kDa.

A therapeutic nanoparticle may, in some embodiments, contain about 10 to about 30 weight percent, in some embodiments about 10 to about 25 weight percent, in some embodiments about 10 to about 20 weight percent, in some embodiments about 10 to about 15 weight percent, in some embodiments about 15 to about 20 weight percent, in some embodiments about 15 to about 25 weight percent, in some embodiments about 20 to about 25 weight percent, in some embodiments about 20 to about 30 weight percent, or in some embodiments about 25 to about 30 weight percent of poly(ethylene)glycol, where the poly(ethylene)glycol may be present as a poly(lactic) acid-poly(ethylene)glycol copolymer, poly(lactic)-co-poly (glycolic) acid-poly(ethylene)glycol copolymer, or poly(ethylene)glycol homopolymer.

In one embodiment, optional small molecule targeting moieties are bonded, e.g., covalently bonded, to the lipid component of the nanoparticle. For example, provided herein is a nanoparticle comprising a polymyxin/colistin antibiotic therapeutic agent (e.g., colistin sulphate), a polymeric matrix comprising functionalized and non-functionalized polymers, optionally a lipid, and optionally a low-molecular weight PSMA targeting ligand, wherein the targeting ligand is bonded, e.g., covalently bonded, to the lipid component of the nanoparticle. In one embodiment, the lipid component that is bonded to the low-molecular weight targeting moiety is of the Formula V. In another embodiment, a target-specific nanoparticle is provided comprising a polymyxin/colistin antibiotic therapeutic agent (e.g., colistin sulphate), a polymeric matrix, DSPE, and a low-molecular weight PSMA targeting ligand, wherein the ligand is bonded, e.g., covalently bonded, to DSPE. For example, the nanoparticle may comprise a polymeric matrix comprising PLGA-DSPE-PEG-Ligand.

### Targeting Moieties

Provided herein are nanoparticles that may include an optional targeting moiety, i.e., a moiety able to bind to or otherwise associate with a biological entity, for example, a membrane component, a cell surface receptor, prostate specific membrane antigen, or the like. A targeting moiety present on the surface of the particle may allow the particle to become localized at a particular targeting site, for instance, a tumor, a disease site, a tissue, an organ, a type of cell, etc. As such, the nanoparticle may then be “target specific.” The drug or other payload may then, in some cases, be released from the particle and allowed to interact locally with the particular targeting site.

In one embodiment, a disclosed nanoparticle includes a targeting moiety that is a low-molecular weight ligand, e.g., a low-molecular weight PSMA ligand. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

For example, a targeting portion may cause the particles to become localized to a tumor (e.g., a solid tumor) a disease site, a tissue, an organ, a type of cell, etc. within the body of a subject, depending on the targeting moiety used. For example, a low-molecular weight PSMA ligand may become localized to a solid tumor, e.g., breast or prostate tumors or cancer cells. The subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

Contemplated targeting moieties may include small molecules. In certain embodiments, the term “small molecule” refers to organic compounds, whether naturally-occurring or artificially created (e.g., via chemical synthesis) that have relatively low molecular weight and that are not proteins, polypeptides, or nucleic acids. Small molecules typically have multiple carbon-carbon bonds. In certain embodiments, small molecules are less than about 2000 g/mol in size. In some embodiments, small molecules are less than about 1500 g/mol or less than about 1000 g/mol. In some embodiments, small molecules are less than about 800 g/mol or less than about 500 g/mol, for example about 100 g/mol to about 600 g/mol, or about 200 g/mol to about 500 g/mol.

For example, a targeting moiety may target prostate cancer tumors, for example a target moiety may be PSMA peptidase inhibitor. These moieties are also referred to herein as “low-molecular weight PSMA ligands.” When compared with expression in normal tissues, expression of prostate specific membrane antigen (PSMA) is at least 10-fold overexpressed in malignant prostate relative to normal tissue, and the level of PSMA expression is further up-regulated as the disease progresses into metastatic phases (Silver et al. 1997, Clin. Cancer Res., 3:81).

In some embodiments, the low-molecular weight PSMA ligand is of the Formulae I, II, III or IV:

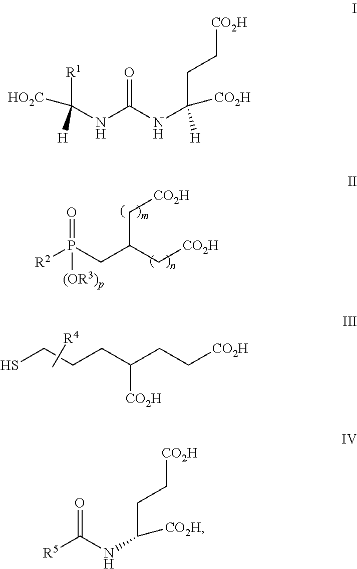

and enantiomers, stereoisomers, rotamers, tautomers, diastereomers, or racemates thereof;

wherein m and n are each, independently, 0, 1, 2 or 3; p is 0 or 1;

R¹, R², R⁴, and R⁵ are each, independently, selected from the group consisting of substituted or unsubstituted alkyl (e.g., C₁₋₁₀-alkyl, C₁₋₆-alkyl, or C₁₋₄-alkyl), substituted or unsubstituted aryl (e.g., phenyl or pyridinyl), and any combination thereof; and R³ is H or C₁₋₆-alkyl (e.g., CH₃).

For compounds of Formulae I, II, III and IV, R¹, R², R⁴ or R⁵ comprise points of attachment to the nanoparticle, e.g., a point of attachment to a polymer that forms part of a disclosed nanoparticle, e.g., PEG. The point of attachment may be formed by a covalent bond, ionic bond, hydrogen bond, a bond formed by adsorption including chemical adsorption and physical adsorption, a bond formed from van der Waals bonds, or dispersion forces. For example, if R¹, R², R⁴, or R⁵ are defined as an aniline or C₁₋₆-alkyl-NH₂ group, any hydrogen (e.g., an amino hydrogen) of these functional groups could be removed such that the low-molecular weight PSMA ligand is covalently bound to the polymeric matrix (e.g., the PEG-block of the polymeric matrix) of the nanoparticle. As used herein, the term “covalent bond” refers to a bond between two atoms formed by sharing at least one pair of electrons.

In particular embodiments of the Formulae I, II, III or IV, R¹, R², R⁴, and R⁵ are each, independently, C₁₋₆-alkyl or phenyl, or any combination of C₁₋₆-alkyl or phenyl, which are independently substituted one or more times with OH, SH, NH₂, or CO₂H, and wherein the alkyl group may be interrupted by N(H), S, or O. In another embodiment, R¹, R², R⁴, and R⁵ are each, independently, CH₂-Ph, (CH₂)₂—SH, CH₂—SH, (CH₂)₂C(H)(NH₂)CO₂H, CH₂C(H)(NH₂)CO₂H, CH(NH₂)CH₂CO₂H, (CH₂)₂C(H)(SH)CO₂H, CH₂—N(H)-Ph, O—CH₂-Ph, or O—(CH₂)₂-Ph, wherein each Ph may be independently substituted one or more times with OH, NH₂, CO₂H, or SH. For these formulae, the NH₂, OH or SH groups serve as the point of covalent attachment to the nanoparticle (e.g., —N(H)—PEG, —O-PEG, or —S-PEG).

In still another embodiment, the low-molecular weight PSMA ligand is selected from the group consisting of

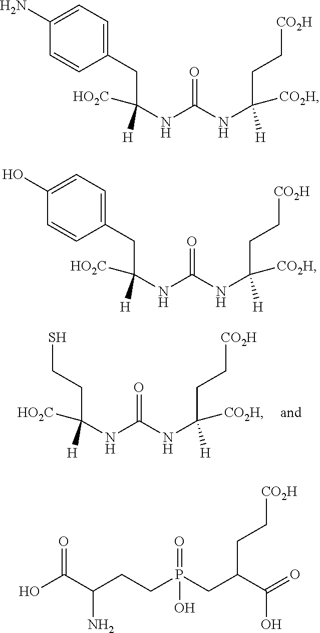

and enantiomers, stereoisomers, rotamers, tautomers, diastereomers, or racemates thereof, and wherein the NH₂, OH, or SH groups serve as the point of covalent attachment to the nanoparticle (e.g., —N(H)—PEG, —O-PEG, or —S-PEG).

In another embodiment, the low-molecular weight PSMA ligand is selected from the group consisting of

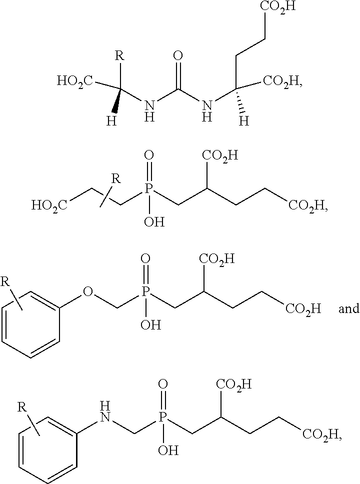

and enantiomers, stereoisomers, rotamers, tautomers, diastereomers, or racemates thereof, wherein R is independently selected from the group consisting of NH₂, SH, OH, CO₂H, C₁₋₆-alkyl that is substituted with NH₂, SH, OH, or CO₂H, and phenyl that is substituted with NH₂, SH, OH, or CO₂H, and wherein R serves as the point of covalent attachment to the nanoparticle (e.g., —N(H)—PEG, —S-PEG, —O-PEG, or CO₂—PEG).

In another embodiment, the low-molecular weight PSMA ligand is selected from the group consisting of

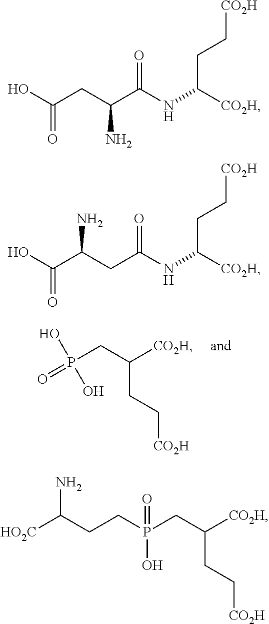

and enantiomers, stereoisomers, rotamers, tautomers, diastereomers, or racemates thereof, wherein the NH₂ or CO₂H groups serve as the point of covalent attachment to the nanoparticle (e.g., —N(H)—PEG, or CO₂—PEG). These compounds may be further substituted with NH₂, SH, OH, CO₂H, C₁₋₆-alkyl that is substituted with NH₂, SH, OH, or CO₂H, or phenyl that is substituted with NH₂, SH, OH or CO₂H, wherein these functional groups can also serve as the point of covalent attachment to the nanoparticle.

In another embodiment, the low-molecular weight PSMA ligand is

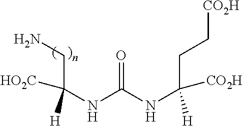

and enantiomers, stereoisomers, rotamers, tautomers, diastereomers, or racemates thereof, wherein n is 1, 2, 3, 4, 5, or 6. For this ligand, the NH₂ group serves as the point of covalent attachment to the nanoparticle (e.g., —N(H)—PEG).

In still another embodiment, the low-molecular weight PSMA ligand is

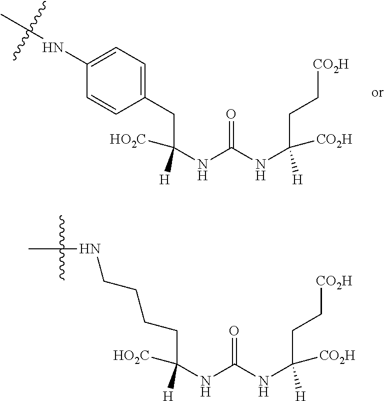 [SMILES: *Nc1ccc(C[C@H](NC(=O)N[C@@H](CCC(=O)O)C(=O)O)C(=O)O)cc1 ; *NCCCC[C@H](NC(=O)N[C@@H](CCC(=O)O)C(=O)O)C(=O)O]

and enantiomers, stereoisomers, rotamers, tautomers, diastereomers, or racemates thereof. Particularly, the butyl-amine compound has the advantage of ease of synthesis, especially because of its lack of a benzene ring. Furthermore, without wishing to be bound by theory, the butyl-amine compound will likely break down into naturally occurring molecules (i.e., lysine and glutamic acid), thereby minimizing toxicity concerns.

In some embodiments, targeting moieties may be used to target cells or tissues damaged by a bacterial infection. In some embodiments, targeting moietites may be used to target bacterium in a tissue. In some embodiments, targeting moieties may be used to target a bacterium in blood, for example, in the case of sepsis.

In some embodiments, the nanoparticles are used to treat diseases and disorders caused by a bacterial infection, for example, syphilis, tuberculosis, salmonella, meningitish, etc. In some embodiments, targeting moieites may be used that target cells damaged by a bacterial infection. In some embodiments, targeting moieties may be used to target a specific tissue, organ or area of the body.

In some embodiments, targeting moieties may target lung tissues. For example, in some embodiments, the nanoparticles of the invention may be used to target lung tissues for the treatment of cystic fibrosis.

In some embodiments, targeting moieties may target slow-growing or dormant bacteria. For example, nanoparticles of the invention may be used for targeting and treating tuberculosis or biofilm-mediated infections.

In some embodiments, targeting moieties may target gram-negative bacteria, including Acinetobacter species, Pseudomonas aeruginosa, Klebsiella species, and Enterobacter species.

For example, contemplated the targeting moieties may include a nucleic acid, polypeptide, glycoprotein, carbohydrate, or lipid. For example, a targeting moiety can be a nucleic acid targeting moiety (e.g. an aptamer, e.g., the A10 aptamer) that binds to a cell type specific marker. In general, an aptamer is an oligonucleotide (e.g., DNA, RNA, or an analog or derivative thereof) that binds to a particular target, such as a polypeptide. In some embodiments, a targeting moiety may be a naturally occurring or synthetic ligand for a cell surface receptor, e.g., a growth factor, hormone, LDL, transferrin, etc. A targeting moiety can be an antibody, which term is intended to include antibody fragments, characteristic portions of antibodies, single chain targeting moieties can be identified, e.g., using procedures such as phage display.

Targeting moieties may be a targeting peptide or targeting peptidomimetic has a length of up to about 50 residues. For example, a targeting moiety may include the amino acid sequence AKERC, CREKA, ARYLQKLN, or AXYLZZLN, wherein X and Z are variable amino acids, or conservative variants or peptidomimetics thereof. In particular embodiments, the targeting moiety is a peptide that includes the amino acid sequence AKERC, CREKA, ARYLQKLN, or AXYLZZLN, wherein X and Z are variable amino acids, and has a length of less than 20, 50 or 100 residues. The CREKA (Cys Arg Glu Lys Ala) peptide or a peptidomimetic thereof peptide or the octapeptide AXYLZZLN are also contemplated as targeting moieties, as well as peptides, or conservative variants or peptidomimetics thereof, that binds or forms a complex with collagen IV, or the targets tissue basement membrane (e.g., the basement membrane of a blood vessel), can be used as a targeting moiety. Exemplary targeting moieties include peptides that target ICAM (intercellular adhesion molecule, e.g., ICAM-1).

Targeting moieties disclosed herein can be, in some embodiments, conjugated to a disclosed polymer or copolymer (e.g., PLA-PEG), and such a polymer conjugate may form part of a disclosed nanoparticle.

In some embodiments, a therapeutic nanoparticle may include a polymer-drug conjugate. For example, a drug may be conjugated to a disclosed polymer or copolymer (e.g., PLA-PEG), and such a polymer-drug conjugate may form part of a disclosed nanoparticle. For example, a disclosed therapeutic nanoparticle may optionally include about 0.2 to about 30 weight percent of a PLA-PEG or PLGA-PEG, wherein the PEG is functionalized with a drug (e.g., PLA-PEG-Drug).

A disclosed polymeric conjugate may be formed using any suitable conjugation technique. For instance, two compounds such as a targeting moiety or drug and a biocompatible polymer (e.g., a biocompatible polymer and a poly(ethylene glycol)) may be conjugated together using techniques such as EDC-NHS chemistry (1-ethyl-3-(3-dimethylaminopropyl) carbodiimide hydrochloride and N-hydroxysuccinimide) or a reaction involving a maleimide or a carboxylic acid, which can be conjugated to one end of a thiol, an amine, or a similarly functionalized polyether. The conjugation of a targeting moiety or drug and a polymer to form a polymer-targeting moiety conjugate or a polymer-drug conjugate can be performed in an organic solvent, such as, but not limited to, dichloromethane, acetonitrile, chloroform, dimethylformamide, tetrahydrofuran, acetone, or the like. Specific reaction conditions can be determined by those of ordinary skill in the art using no more than routine experimentation.

In another set of embodiments, a conjugation reaction may be performed by reacting a polymer that comprises a carboxylic acid functional group (e.g., a poly(ester-ether) compound) with a polymer or other moiety (such as a targeting moiety or drug) comprising an amine. For instance, a targeting moiety, such as a low-molecular weight PSMA ligand, or a drug, such as dasatinib, may be reacted with an amine to form an amine-containing moiety, which can then be conjugated to the carboxylic acid of the polymer. Such a reaction may occur as a single-step reaction, i.e., the conjugation is performed without using intermediates such as N-hydroxysuccinimide or a maleimide. In some embodiments, a drug may be reacted with an amine-containing linker to form an amine-containing drug, which can then be conjugated to the carboxylic acid of the polymer as described above. The conjugation reaction between the amine-containing moiety and the carboxylic acid-terminated polymer (such as a poly(ester-ether) compound) may be achieved, in one set of embodiments, by adding the amine-containing moiety, solubilized in an organic solvent such as (but not limited to) dichloromethane, acetonitrile, chloroform, tetrahydrofuran, acetone, formamide, dimethylformamide, pyridines, dioxane, or dimethylsulfoxide, to a solution containing the carboxylic acid-terminated polymer. The carboxylic acid-terminated polymer may be contained within an organic solvent such as, but not limited to, dichloromethane, acetonitrile, chloroform, dimethylformamide, tetrahydrofuran, or acetone. Reaction between the amine-containing moiety and the carboxylic acid-terminated polymer may occur spontaneously, in some cases. Unconjugated reactants may be washed away after such reactions, and the polymer may be precipitated in solvents such as, for instance, ethyl ether, hexane, methanol, or ethanol. In certain embodiments, a conjugate may be formed between an alcohol-containing moiety and carboxylic acid functional group of a polymer, which can be achieved similarly as described above for conjugates of amines and carboxylic acids.

As a specific example, a low-molecular weight PSMA ligand may be prepared as a targeting moiety in a particle as follows. Carboxylic acid modified poly(lactide-co-glycolide) (PLGA-COOH) may be conjugated to an amine-modified heterobifunctional poly(ethylene glycol) (NH₂—PEG-COOH) to form a copolymer of PLGA-PEG-COOH. By using an amine-modified low-molecular weight PSMA ligand (NH₂-Lig), a triblock polymer of PLGA-PEG-Lig may be formed by conjugating the carboxylic acid end of the PEG to the amine functional group on the ligand. The multiblock polymer can then be used, for instance, as discussed below, e.g., for therapeutic applications.

### Nanoparticles

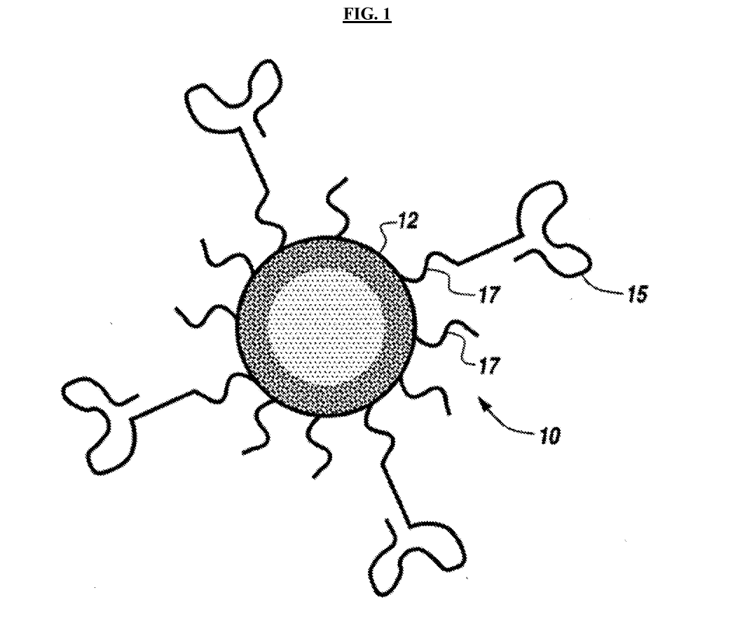

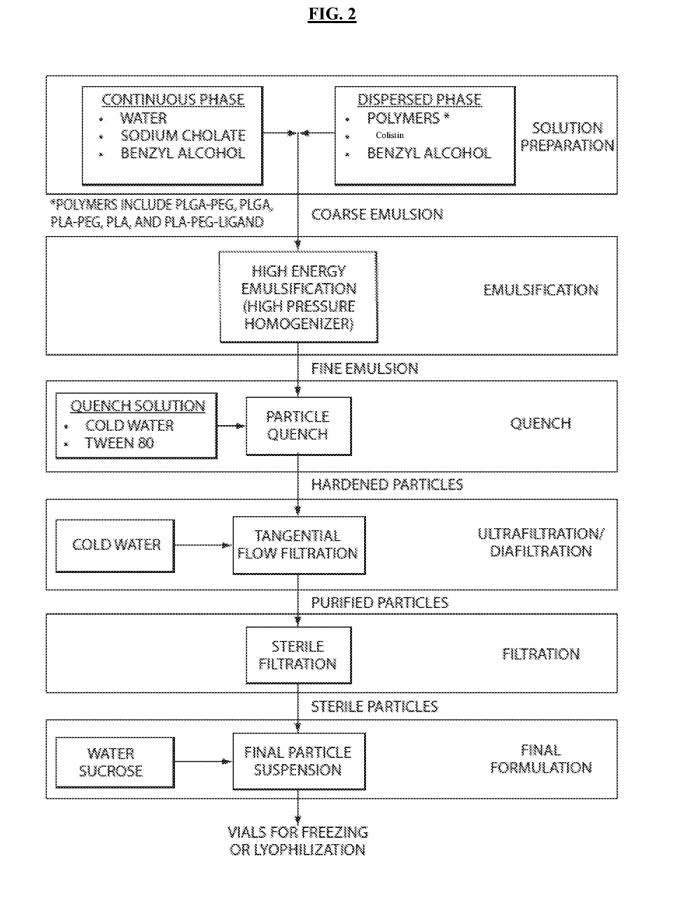

Referring now to FIG. 1, a contemplated nanoparticle is shown as a non-limiting example. In this figure, a polymeric conjugate of the disclosure is used to form a nanoparticle 10. The polymer forming nanoparticle 10 includes a low-molecular weight ligand 15, present on the surface of the particle, and a biocompatible portion 17. In some cases, as shown here, targeting moiety 15 may be conjugated to biocompatible portion 17. However, not all of biocompatible portion 17 is shown conjugated to targeting moiety 15. For instance, in some cases, nanoparticles such as nanoparticle 10 may be formed using a first polymer comprising biocompatible portion 17 and low-molecular weight ligand 15, and a second polymer comprising biocompatible portion 17 but not targeting moiety 15. By controlling the ratio of the first and second polymers, nanoparticles having different properties may be formed, and in some cases, libraries of such nanoparticles may be formed. In addition, nanoparticle 10 contains polymyxin/colistin antibiotic therapeutic agent 12. In some cases, therapeutic agent 12 may be associated with the surface of, encapsulated within, surrounded by, or dispersed throughout the nanoparticle. In another embodiment, the therapeutic agent is encapsulated within the hydrophobic core of the nanoparticle.

Disclosed nanoparticles may be stable (e.g., retain substantially all active agent) for example in a solution that may contain a saccharide, for at least about 3 days, about 4 days or at least about 5 days at room temperature, or at 25° C.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

Nanoparticles may have controlled release properties, e.g., may be capable of delivering an amount of a polymyxin/colistin antibiotic therapeutic agent to a patient, e.g., to specific site in a patient, over an extended period of time, e.g., over 1 day, 1 week, or more.

In some embodiments, after administration to a subject or patient of a disclosed nanoparticle or a composition that includes a disclosed nanoparticle, the peak plasma concentration (Cₘₐₓ) of the polymyxin/colistin antibiotic therapeutic agent in the patient is substantially higher as compared to a Cₘₐₓ of the therapeutic agent if administered alone (e.g., not as part of a nanoparticle).

In another embodiment, a disclosed nanoparticle including a therapeutic agent, when administered to a subject, may have a tₘₐₓ of therapeutic agent substantially longer as compared to a tₘₐₓ of the therapeutic agent administered alone.

Libraries of such particles may also be formed. For example, by varying the ratios of the two (or more) polymers within the particle, these libraries can be useful for screening tests, high-throughput assays, or the like. Entities within the library may vary by properties such as those described above, and in some cases, more than one property of the particles may be varied within the library. Accordingly, one embodiment is directed to a library of nanoparticles having different ratios of polymers with differing properties. The library may include any suitable ratio(s) of the polymers.

In some embodiments, the biocompatible polymer is a hydrophobic polymer. Non-limiting examples of biocompatible polymers include polylactide, polyglycolide, and/or poly(lactide-co-glycolide).

In a different embodiment, this disclosure provides for a nanoparticle comprising 1) a polymeric matrix; 2) optionally, an amphiphilic compound or layer that surrounds or is dispersed within the polymeric matrix forming a continuous or discontinuous shell for the particle; 3) a non-functionalized polymer that may form part of the polymeric matrix, and 4) optionally, a low molecular weight ligand that binds to a target protein conjugate such as PSMA, covalently attached to a polymer, which may form part of the polymeric matrix. For example, an amphiphilic layer may reduce water penetration into the nanoparticle, thereby enhancing drug encapsulation efficiency and slowing drug release.

As used herein, the term “amphiphilic” refers to a property where a molecule has both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. In some embodiments, the amphiphilic compound can be, but is not limited to, one or a plurality of the following: naturally derived lipids, surfactants, or synthesized compounds with both hydrophilic and hydrophobic moieties.

Specific examples of amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-γ-alkyl phospholipids. Examples of phospholipids include, but are not limited to, phosphatidylcholines such as dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used.

In a particular embodiment, an amphiphilic component that can be used to form an amphiphilic layer is lecithin, and, in particular, phosphatidylcholine. Lecithin is an amphiphilic lipid and, as such, forms a phospholipid bilayer having the hydrophilic (polar) heads facing their surroundings, which are oftentimes aqueous, and the hydrophobic tails facing each other. Lecithin has an advantage of being a natural lipid that is available from, e.g., soybean, and already has FDA approval for use in other delivery devices. In addition, a mixture of lipids such as lethicin is more advantageous than one single pure lipid.

In certain embodiments a disclosed nanoparticle has an amphiphilic monolayer, meaning the layer is not a phospholipid bilayer, but exists as a single continuous or discontinuous layer around, or within, the nanoparticle. The amphiphilic layer is “associated with” the nanoparticle, meaning it is positioned in some proximity to the polymeric matrix, such as surrounding the outside of the polymeric shell, or dispersed within the polymers that make up the nanoparticle.

### Therapeutic Agents

The polymyxin/colistin antibiotic therapeutic agent may include any antibiotic, e.g. such as from the polymyxin/colistin family. Polymyxins, a group of cationic polypeptide antibiotics, consist of five chemically different compounds (Polymyxins A-E). Colistin is a cationic, multicomponent lipopeptide consisting of a cyclic heptapeptide with a tripeptide side chain acylated at the N terminus by a fatty acid. The two major components of colistin are colistin A (polymyxins E₁) and colistin B (polymyxins E₂). The cationic polypeptides of colistin and polymyxin B interact with anionic lipopolysaccharide (LPS) molecules in the outer membrane of gram-negative bacteria, leading to displacement of calcium (Ca²⁺) and magnesium (Mg²⁺), which stabilize the LPS membrane, thus causing derangement of the cell membrane. This results in an increase in the permeability of the cell membrane, leakage of cell contents, and ultimately cell death. Colistin also has potent anti-endotoxin activity. The endotoxin of a gram-negative organism is the lipid A portion of a LPS molecule and colistin binds and neutralizes this LPS molecule. Alternative forms of an antibiotic from the polymyxin/colistin family may be used, for example, pharmaceutically acceptable salt forms, free base forms, sulfates, hydrates, isomers, and prodrugs thereof. For example, either form of colistin may be used: colistin sulfate and CMS (colistimethate sodium, colistin methanesulfate, pentasodium colistimethanesulfate, and colistin sulfonyl methate). CMS is an inactive prodrug of colistin and is less potent and less toxic than colistin sulphate. Polymyxin B and colistin differ only in their amino acid components. Polymyxin B and colistin are known antimicrobials. Both colistin and polymyxin B have potent, concentration-dependent killing capacity against MDR gram-negative bacteria. Colistin has bactericidal activity against most gram-negative aerobic bacilli, including Acinetobacter species (MIC₉₀≤2 mg/L), P. aeruginosa (MIC₉₀≤4 mg/L), K. pneumoniae (MIC₉₀≤1 mg/L), E. coli (MIC₉₀≤2 mg/L), and Enterobacter spp (MIC₅₀≤1 mg/L). It also may be active against Salmonella spp (MIC₉₀≤1 mg/L), Shigella spp (MIC₉₀≤0.5 mg/L), Citrobacter spp (MIC₉₀≤1 mg/L), Yersinia pseudotuberculosis, Haemophilus influenzae, and several mycobacterial species. Providentia spp, Serratia spp, and Brucella spp may be resistant to colistin.

The polymyxin/colistin antibiotic therapeutic agent may disorganize the structure or inhibit the function of bacterial membranes. The integrity of the cytoplasmic and outer membranes is vital to bacteria, and compounds that disorganize the membranes rapidly kill the cells.

In one set of embodiments, the payload is a drug, or antibiotic, or a combination of more than one drug or antibiotic. Such particles may be useful, for example, in embodiments where a targeting moiety may be used to direct a particle containing a drug to a particular localized location within a subject, e.g., to allow localized delivery of the drug to occur.

### Pharmaceutical Formulations

Nanoparticles disclosed herein may be combined with pharmaceutically acceptable carriers to form a pharmaceutical composition, according to another aspect. As would be appreciated by one of skill in this art, the carriers may be chosen based on the route of administration as described below, the location of the target issue, the drug being delivered, the time course of delivery of the drug, etc.

The pharmaceutical compositions can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (0 absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

It will be appreciated that the exact dosage of a nanoparticle containing a antibiotic therapeutic agent is chosen by the individual physician in view of the patient to be treated, in general, dosage and administration are adjusted to provide an effective amount of the antibiotic therapeutic agent nanoparticle to the patient being treated. As used herein, the “effective amount” of a nanoparticle containing a antibiotic therapeutic agent refers to the amount necessary to elicit the desired biological response. As will be appreciated by those of ordinary skill in this art, the effective amount of a nanoparticle containing a polymyxin/colistin atherapeutic agent may vary depending on such factors as the desired biological endpoint, the drug to be delivered, the target tissue, the route of administration, etc. For example, the effective amount of a nanoparticle containing a antibiotic therapeutic agent might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The nanoparticles may be formulated in dosage unit form for ease of administration and uniformity of dosage. The expression “dosage unit form” as used herein refers to a physically discrete unit of nanoparticle appropriate for the patient to be treated. It will be understood, however, that the total daily usage of the compositions will be decided by the attending physician within the scope of sound medical judgment. For any nanoparticle, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

In an embodiment, compositions disclosed herein may include less than about 10 ppm of palladium, or less than about 8 ppm, or less than about 6 ppm of palladium. For example, provided here is a composition that includes nanoparticles having a polymeric conjugate wherein the composition has less than about 10 ppm of palladium.

In some embodiments, a composition suitable for freezing is contemplated, including nanoparticles disclosed herein and a solution suitable for freezing, e.g., a sugar such as a mono, di, or poly saccharide, e.g., sucrose and/or a trehalose, and/or a salt and/or a cyclodextrin solution is added to the nanoparticle suspension. The sugar (e.g., sucrose or trehalose) may act, e.g., as a cryoprotectant to prevent the particles from aggregating upon freezing. For example, provided herein is a nanoparticle formulation comprising a plurality of disclosed nanoparticles, sucrose, an ionic halide, and water; wherein the nanoparticles/sucrose/water/ionic halide is about 3-40%/10-40%/20-95%/0.1-10% (w/w/w/w) or about 5-10%/10-15%/80-90%/1-10% (w/w/w/w). For example, such solution may include nanoparticles as disclosed herein, about 5% to about 20% by weight sucrose and an ionic halide such as sodium chloride, in a concentration of about 10-100 mM. In another example, provided herein is a nanoparticle formulation comprising a plurality of disclosed nanoparticles, trehalose, cyclodextrin, and water; wherein the nanoparticles/trehalose/water/cyclodextrin is about 3-40%/1-25%/20-95%/1-25% (w/w/w/w) or about 5-10%/1-25%/80-90%/10-15% (w/w/w/w).

For example, a contemplated solution may include nanoparticles as disclosed herein, about 1% to about 25% by weight of a disaccharide such as trehalose or sucrose (e.g., about 5% to about 25% trehalose or sucrose, e.g. about 10% trehalose or sucrose, or about 15% trehalose or sucrose, e.g. about 5% sucrose) by weight) and a cyclodextrin such as β-cyclodextrin, in a concentration of about 1% to about 25% by weight (e.g. about 5% to about 20%, e.g. 10% or about 20% by weight, or about 15% to about 20% by weight cyclodextrin). Contemplated formulations may include a plurality of disclosed nanoparticles (e.g. nanoparticles having PLA-PEG and an active agent), and about 2% to about 15 wt % (or about 4% to about 6 wt %, e.g. about 5 wt %) sucrose and about 5 wt % to about 20% (e.g. about 7% wt percent to about 12 wt %, e.g. about 10 wt %) of a cyclodextrin, e.g., HPbCD).

The present disclosure relates in part to lyophilized pharmaceutical compositions that, when reconstituted, have a minimal amount of large aggregates. Such large aggregates may have a size greater than about 0.5 μm, greater than about 1 μm, or greater than about 10 μm, and can be undesirable in a reconstituted solution. Aggregate sizes can be measured using a variety of techniques including those indicated in the U.S. Pharmacopeia at 32 <788>, hereby incorporated by reference. The tests outlined in USP 32 <788> include a light obscuration particle count test, microscopic particle count test, laser diffraction, and single particle optical sensing. In one embodiment, the particle size in a given sample is measured using laser diffraction and/or single particle optical sensing.

The USP 32 <788> by light obscuration particle count test sets forth guidelines for sampling particle sizes in a suspension. For solutions with less than or equal to 100 mL, the preparation complies with the test if the average number of particles present does not exceed 6000 per container that are ≥10 μm and 600 per container that are ≥25 μm.

As outlined in USP 32 <788>, the microscopic particle count test sets forth guidelines for determining particle amounts using a binocular microscope adjusted to 100±10× magnification having an ocular micrometer. An ocular micrometer is a circular diameter graticule that consists of a circle divided into quadrants with black reference circles denoting 10 μm and 25 μm when viewed at 100× magnification. A linear scale is provided below the graticule. The number of particles with reference to 10 μm and 25 μm are visually tallied. For solutions with less than or equal to 100 mL, the preparation complies with the test if the average number of particles present does not exceed 3000 per container that are ≥10 μm and 300 per container that are ≥25 μm.

In some embodiments, a 10 mL aqueous sample of a disclosed composition upon reconstitution comprises less than 600 particles per ml having a size greater than or equal to 10 microns; and/or less than 60 particles per ml having a size greater than or equal to 25 microns.

Dynamic light scattering (DLS) may be used to measure particle size, but it relies on Brownian motion so the technique may not detect some larger particles. Laser diffraction relies on differences in the index of refraction between the particle and the suspension media. The technique is capable of detecting particles at the sub-micron to millimeter range. Relatively small (e.g., about 1-5 weight %) amounts of larger particles can be determined in nanoparticle suspensions. Single particle optical sensing (SPOS) uses light obscuration of dilute suspensions to count individual particles of about 0.5 μm. By knowing the particle concentration of the measured sample, the weight percentage of aggregates or the aggregate concentration (particles/mL) can be calculated.

Formation of aggregates can occur during lyophilization due to the dehydration of the surface of the particles. This dehydration can be avoided by using lyoprotectants, such as disaccharides, in the suspension before lyophilization. Suitable disaccharides include sucrose, lactulose, lactose, maltose, trehalose, or cellobiose, and/or mixtures thereof. Other contemplated disaccharides include kojibiose, nigerose, isomaltose, β,β-trehalose, α,β-trehalose, sophorose, laminaribiose, gentiobiose, turanose, maltulose, palatinose, gentiobiulose, mannobiase, melibiose, melibiulose, rutinose, rutinulose, and xylobiose. Reconstitution shows equivalent DLS size distributions when compared to the starting suspension. However, laser diffraction can detect particles of >10 μm in size in some reconstituted solutions. Further, SPOS also may detect >10 μm sized particles at a concentration above that of the FDA guidelines (10⁴-10⁵ particles/mL for >10 μm particles).

In some embodiments, one or more ionic halide salts may be used as an additional lyoprotectant to a sugar, such as sucrose, trehalose or mixtures thereof. Sugars may include disaccharides, monosaccharides, trisaccharides, and/or polysaccharides, and may include other excipients, e.g. glycerol and/or surfactants. Optionally, a cyclodextrin may be included as an additional lyoprotectant. The cyclodextrin may be added in place of the ionic halide salt. Alternatively, the cyclodextrin may be added in addition to the ionic halide salt.

Suitable ionic halide salts may include sodium chloride, calcium chloride, zinc chloride, or mixtures thereof. Additional suitable ionic halide salts include potassium chloride, magnesium chloride, ammonium chloride, sodium bromide, calcium bromide, zinc bromide, potassium bromide, magnesium bromide, ammonium bromide, sodium iodide, calcium iodide, zinc iodide, potassium iodide, magnesium iodide, or ammonium iodide, and/or mixtures thereof. In one embodiment, about 1 to about 15 weight percent sucrose may be used with an ionic halide salt. In one embodiment, the lyophilized pharmaceutical composition may comprise about 10 to about 100 mM sodium chloride. In another embodiment, the lyophilized pharmaceutical composition may comprise about 100 to about 500 mM of divalent ionic chloride salt, such as calcium chloride or zinc chloride. In yet another embodiment, the suspension to be lyophilized may further comprise a cyclodextrin, for example, about 1 to about 25 weight percent of cyclodextrin may be used.

A suitable cyclodextrin may include α-cyclodextrin, β-cyclodextrin, γ-cyclodextrin, or mixtures thereof. Exemplary cyclodextrins contemplated for use in the compositions disclosed herein include hydroxypropyl-β-cyclodextrin (HPbCD), hydroxyethyl-β-cyclodextrin, sulfobutylether-β-cyclodextrin, methyl-β-cyclodextrin, dimethyl-β-cyclodextrin, carboxymethyl-β-cyclodextrin, carboxymethyl ethyl-β-cyclodextrin, diethyl-β-cyclodextrin, tri-O-alkyl-β-cyclodextrin, glocosyl-β-cyclodextrin, and maltosyl-β-cyclodextrin. In one embodiment, about 1 to about 25 weight percent trehalose (e.g. about 10% to about 15%, e.g. 5 to about 20% by weight) may be used with cyclodextrin. In one embodiment, the lyophilized pharmaceutical composition may comprise about 1 to about 25 weight percent β-cyclodextrin. An exemplary composition may comprise nanoparticles comprising PLA-PEG, an active/therapeutic agent, about 4% to about 6% (e.g. about 5% wt percent) sucrose, and about 8 to about 12 weight percent (e.g. about 10 wt. %) HPbCD.

In one aspect, a lyophilized pharmaceutical composition is provided comprising disclosed nanoparticles, wherein upon reconstitution of the lyophilized pharmaceutical composition at a nanoparticle concentration of about 50 mg/mL, in less than or about 100 mL of an aqueous medium, the reconstituted composition suitable for parenteral administration comprises less than 6000, such as less than 3000, microparticles of greater than or equal to 10 microns; and/or less than 600, such as less than 300, microparticles of greater than or equal to 25 microns.

The number of microparticles can be determined by means such as the USP 32 <788> by light obscuration particle count test, the USP 32 <788> by microscopic particle count test, laser diffraction, and single particle optical sensing.

In an aspect, a pharmaceutical composition suitable for parenteral use upon reconstitution is provided comprising a plurality of therapeutic particles each comprising a copolymer having a hydrophobic polymer segment and a hydrophilic polymer segment; an active agent; a sugar; and a cyclodextrin.

For example, the copolymer may be poly(lactic) acid-block-poly(ethylene)glycol copolymer. Upon reconstitution, a 100 mL aqueous sample may comprise less than 6000 particles having a size greater than or equal to 10 microns; and less than 600 particles having a size greater than or equal to 25 microns.

The step of adding a disaccharide and an ionic halide salt may comprise adding about 5 to about 15 weight percent sucrose or about 5 to about 20 weight percent trehalose (e.g., about 10 to about 20 weight percent trehalose), and about 10 to about 500 mM ionic halide salt. The ionic halide salt may be selected from sodium chloride, calcium chloride, and zinc chloride, or mixtures thereof. In an embodiment, about 1 to about 25 weight percent cyclodextrin is also added.

In another embodiment, the step of adding a disaccharide and a cyclodextrin may comprise adding about 5 to about 15 weight percent sucrose or about 5 to about 20 weight percent trehalose (e.g., about 10 to about 20 weight percent trehalose), and about 1 to about 25 weight percent cyclodextrin. In an embodiment, about 10 to about 15 weight percent cyclodextrin is added. The cyclodextrin may be selected from α-cyclodextrin, β-cyclodextrin, γ-cyclodextrin, or mixtures thereof.

In another aspect, a method of preventing substantial aggregation of particles in a pharmaceutical nanoparticle composition is provided comprising adding a sugar and a salt to the lyophilized formulation to prevent aggregation of the nanoparticles upon reconstitution. In an embodiment, a cyclodextrin is also added to the lyophilized formulation. In yet another aspect, a method of preventing substantial aggregation of particles in a pharmaceutical nanoparticle composition is provided comprising adding a sugar and a cyclodextrin to the lyophilized formulation to prevent aggregation of the nanoparticles upon reconstitution.

A contemplated lyophilized composition may have a therapeutic particle concentration of greater than about 40 mg/mL. The formulation suitable for parenteral administration may have less than about 600 particles having a size greater than 10 microns in a 10 mL dose. Lyophilizing may comprise freezing the composition at a temperature of greater than about −40° C., or e.g. less than about −30° C., forming a frozen composition; and drying the frozen composition to form the lyophilized composition. The step of drying may occur at about 50 mTorr at a temperature of about −25 to about −34° C., or about −30 to about −34° C.

### Methods of Treatment

In some embodiments, targeted nanoparticles may be used to treat, alleviate, ameliorate, relieve, delay onset of, inhibit progression of, reduce severity of, and/or reduce incidence of one or more symptoms or features of a disease, disorder, and/or condition. In some embodiments, targeted nanoparticles may be used to treat infections, e.g., infections caused by Gram-negative bacteria. Gram-negative bacteria cause infections including pneumonia, bloodstream infections, wound or surgical site infections, and meningitis in healthcare settings. Gram-negative bacteria are resistant to multiple drugs and are increasingly resistant to most available antibiotics. In certain embodiments, targeted nanoparticles may be used to treat any infection involving a bacterium, or at least one type of bacterium

As used herein, the term “infection” may include bacterial, microbial, or fungal infections. In some embodiments the infection is an invasion of an organism's body tissues by disease-causing agents, their multiplication, and the reaction of host tissues to these organisms an the toxins they produce. Infection begins when an organism successfully colonizes by entering the body, growing and multiplying. While a few organisms can grow at the initial site of entry, many migrate and cause systemic infection in different organs. Some pathogens grow within the host cells (intracellular) whereas others grow freely in bodily fluids. Microorganisms can cause tissue damage by releasing a variety of toxins or destructive enzymes. For example, Clostridium tetani releases a toxin that paralyzes muscles, and Staphylococcus releases toxins that produce shock and sepsis.

Infections may include infectious diseases. For example, infectious diseases may be, or be caused by: Escherichia coli, Salmonella, Helicobacter pyloi, Neisseria gonorrhoeae, Neisseria meningitides, Staphylococcus aureus, Streptococcal bacteria, MRSA, Lyme's disease, syphilis, Hansen's disease, tuberculosis, tetanus, Colstridium, Botulism, Bubonic plague, Cholera, Acinetobacter baumannii, meningitis, gonorrhea, typhoid, Campylobacter, and diphtheria.

In some embodiments, nanoparticles are used as an antimicrobial, to treat related diseases and disorders. As an antimicrobial, microorganisms are killed, or their growth is inhibited. In some embodiments, the nanoparticles are used as an antimicrobial medicines to treat infection (antimicrobial chemotherapy). In some embodiments, the nanoparticles are used as antimicrobial medicines to prevent infection (antimicrobial prophylaxis). For example, nanoparticles of the invention may be used to treat diseases and disorders associated with Pseudomonas aeruginosa (e.g., inflammation and sepsis).

In some embodiments, nanoparticles are used as antifungals. As an antifungal, the nanoparticles kill or prevent further growth of fungi. For example, they may be used for the treatment for infections such as athlete's foot, ringworm and thrush. They kill off the fungal organism without dangerous effects on the host. For example, the nanoparticles may be used to inhibited the growth of Candida albicans and Saccharomyces cerevisiae.

In one aspect, a method for the treatment of an infection (e.g., tuberculosis, cystic fibrosis) is provided. In some embodiments, the treatment of an infection comprises administering a therapeutically effective amount of inventive targeted particles to a subject in need thereof, in such amounts and for such time as is necessary to achieve the desired result. In certain embodiments, a “therapeutically effective amount” of an inventive targeted particle is that amount effective for treating, alleviating, ameliorating, relieving, delaying onset of, inhibiting progression of, reducing severity of, and/or reducing incidence of one or more symptoms or features of cancer.

In one aspect, a method for administering inventive compositions to a subject suffering from an infection (e.g., tuberculosis, cystic fibrosis) is provided. In some embodiments, particles may be administered to a subject in such amounts and for such time as is necessary to achieve the desired result (i.e., treatment of an infection). In certain embodiments, a “therapeutically effective amount” of an inventive targeted particle is that amount effective for treating, alleviating, ameliorating, relieving, delaying onset of, inhibiting progression of, reducing severity of, and/or reducing incidence of one or more symptoms or features of an infection.

Also provided herein are methods of administering to a patient a nanoparticle disclosed herein including an active agent, wherein, upon administration to a patient, such nanoparticles substantially reduces the volume of distribution and/or substantially reduces free Cₘₐₓ, as compared to administration of the agent alone (i.e., not as a disclosed nanoparticle).

U.S. Pat. No. 8,206,747, issued Jun. 26, 2012, entitled “Drug Loaded Polymeric Nanoparticles and Methods of Making and Using Same” is hereby incorporated by reference in its entirety.

### EXAMPLES

The invention now being generally described, it will be more readily understood by reference to the following examples which are included merely for purposes of illustration of certain aspects and embodiments, and are not intended to limit the invention in any way.

### Example 1: Preparation of Colistin-Containing Nanoparticles

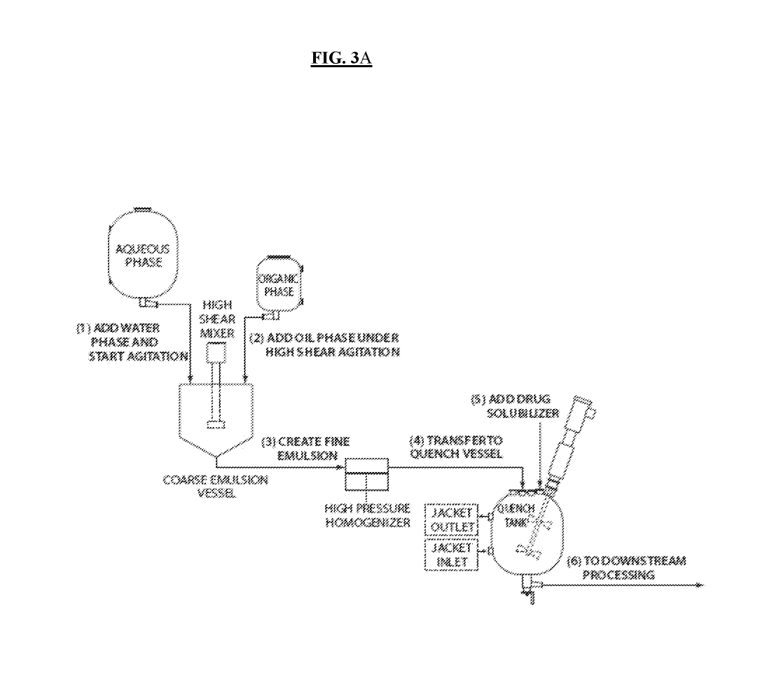

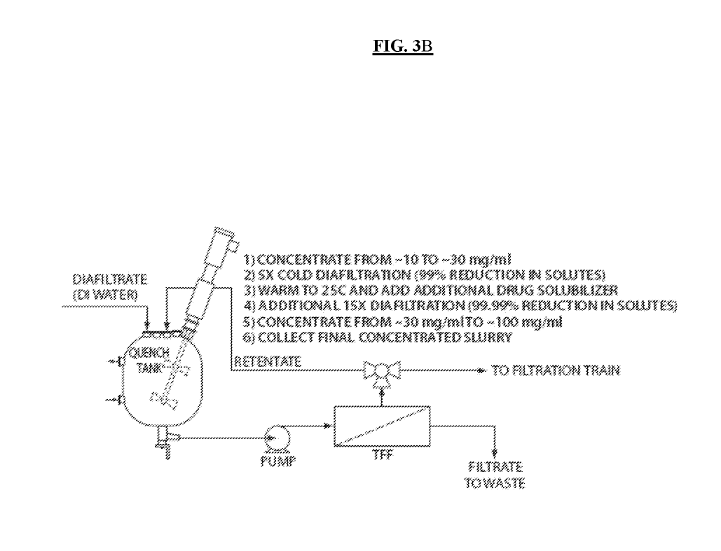

Nanoparticles were prepared using the general process outlined in FIGS. 2 and 3. Three general strategies were applied for preparation of nanoparticles (NPs). In the first strategy, the hydrophobic acids as counter ions were dissolved in an oil phase together with PLA-PEG polymer. The colistin sulphate (CS) was added to a water phase as a water solution. In addition, a calculated amount of NaOH was added to the water phase for complete neutralization of colistin amines. The oil phase was mixed with the water phase and processed to obtain a fine emulsion. The emulsion was quenched in buffer solution at pH 6-8 or in pure water. In the second strategy, the hydrophobic acids as counter ions were dissolved in an oil phase together with PLA-PEG polymer. The colistin sulphate was added to a water phase as a water solution. The oil phase was mixed with the water phase and processed to obtain a fine emulsion. The emulsion was quenched in buffer solution at pH 6-8. In the third strategy, PLA-PEG was dissolved in an oil phase and processed to obtain a fine emulsion. Calculated amounts of sodium salts of hydrophobic acids dissolved in water were added to the fine emulsion followed by addition of colistin sulphate dissolved in water. The resulting mixture was quenched in pure water or buffer solutions at pH 6-8. Further examples provide the preparation procedures of nanoparticles in more detail.

All strategies resulted to encapsulation of colistin into nanoparticles. The first and third strategies resulted in nanoparticles with similar loadings and in vitro release (IVR) profiles (i.e. for decanoic acid as a counter ion the loading was around 10% and the IVR at 24 hours was around 15-20%). The second strategy resulted in lower loadings and significantly slower release profiles (i.e. for decanoic acid as a counter ion the loading was around 5% and the IVR at 24 hours was around 5%). Such results could be explained by the fact that colistin should form an ion pair in the water phase and transfer to fine emulsion in this form. Incomplete neutralization of counter ions during quenching in buffer solutions during the second strategy results in poor encapsulation of colisitin and higher amounts of hydrophobic acids in final NPs, which facilitates slower release. Ratios between counter ions and colistin have an optimum in the range between 5/1 and 10/1. Above and below these values poor encapsulation of drug was observed with practically no impact on release rate.

Strategy 1 was used as a baseline for preparation of majority of formulations.

### Example 2: Preparation of Colistin-Containing Nanoparticles-Counter Ion Screening

Different counter ions (CI) screened for formation of nanoparticles at a 5/1 CS/CI ratio included aliphatic acids (C5-C18), aromatic acids (xinaphoic, pamoic, naphtoic), and cholates (cholic and deoxycholic acid).

Detailed formulations are as follows. For decanoic acid formulation S, the oil phase was prepared with 1.0 g of polylactic acid-block-polyethylene glycol diblock-copolymer (PLA-PEG) (10 kDa PLA-SkDa PEG), which was dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. Decanoic acid solution was prepared by adding 0.123 g of decanoic acid (DA) and 0.05 ml NaOH solution (40% w/w) to 0.45 ml of deionized water. The water phase was prepared by dissolving 4% BA and 0.4% of Brij 5100 in deionized water, and then cooling the solution to approximately 2° C. For processing, 5 g of the oil phase solution was dumped into the water phase under strong mixing. The resulting coarse emulsion was processed on a Microfluidics 110P device at 10,000 psi resulting in a fine emulsion. Under vigorous mixing, the decanoic acid solution was added to the fine emulsion, followed by the colistin sulphate solution. The final emulsion was incubated for 1 minute on ice, and dumped in to 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of colistin (10%).

For the deoxycholic acid formulation, the oil phase was prepared with 1.0 g of polylactic acid-block-polyethylene glycol diblock-copolymer (PLA-PEG) (10 kDa PLA-5 kDa PEG), which was dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. Deoxycholic acid solution was prepared by adding 0.303 g of deoxycholic acid (DA) and 0.05 ml NaOH solution (40% w/w) to 0.45 ml of deionized water. The water phase was prepared by dissolving 4% BA and 0.4% of Brij S100 in deionized water, and then cooling the solution to approximately 2° C. For processing, 5 g of the oil phase solution was dumped into the water phase under strong mixing. The resulting coarse emulsion was processed on a Microfluidics 110P device at 10,000 psi resulting in a fine emulsion. Under vigorous mixing, the deoxycholic acid solution was added to the fine emulsion, followed by the colistin sulphate solution. The final emulsion was incubated for 1 minute on ice, and dumped into 300 g of cold water (<2° C.) under mixing. 28 g of Tween 80 solution in water (35% w/w) was added and mixed for 3 minutes. Downstream, the organic solvents, processing aids, non-bound drug, and deoxycholic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of colistin (10%).

For the pamoic acid formulation, the oil phase was prepared with 1.0 g of polylactic acid-block-polyethylene glycol diblock-copolymer (PLA-PEG) (10 kDa PLA-5 kDa PEG), which was dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. Pamoic acid solution was prepared by adding 0.139 g of pamoic acid (PA) and 0.05 ml NaOH solution (40% w/w) to 0.45 ml of deionized water. The water phase was prepared by dissolving 4% BA and 0.4% of Brij S100 in deionized water, and then cooling the solution to approximately 2° C. For processing, 5 g of the oil phase solution was dumped into the water phase under strong mixing. The resulting coarse emulsion was processed on a Microfluidics 110P device at 10,000 psi resulting in a fine emulsion. Under vigorous mixing, the panoic acid solution was added to the fine emulsion, followed by the colistin sulphate solution. The final emulsion was incubated for 1 minute on ice, and dumped into 300 g of cold water (<2° C.) under mixing. 28 g of Tween 80 solution in water (35% w/w) was added and mixed for 3 minutes. Downstream, the organic solvents, processing aids, non-bound drug, and pamoic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 40 mg of colistin (4%).

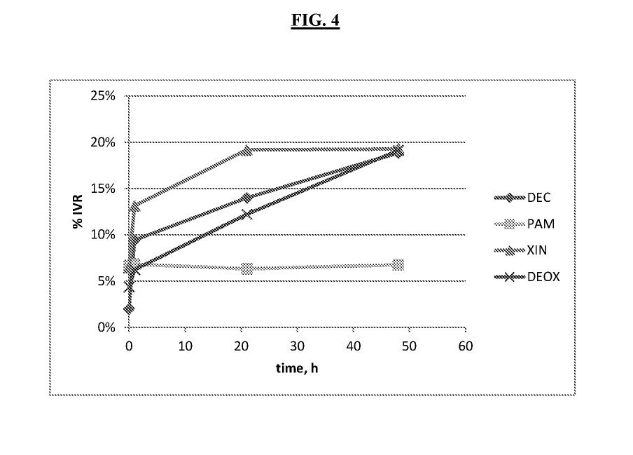

Aromatic acids showed relatively poor potential for encapsulation of colistin. The loadings did not exceed 5% and were typically in the range of 3-4%. Due to low solubility of cholic and deoxycholic acid in oil phase only strategy 3 could be applied for preparation of nanoparticles. NPs with deoxycholic acid showed relatively high loadings (around 10%). For aliphatic acids the particles showed optimum loading at the range C9-C12. Acids of lower hydrophobicity did not provide good encapsulation into NPs whereas the acids with longer aliphatic tails (i.e. oleic acid) showed relatively high burst release (>20%). The in vitro release rates for formulations are presented in FIG. 4.

### Example 3: Preparation of Colistin-Containing Nanoparticles-Decanoic Acid Formulations

The use of decanoic acid was found to be optimal for further investigations. Optimization of colistin sulphate NPs (CS-NPs) with decanoic acid revealed that two different formulations could be obtained depending on theoretical loading parameters used for preparation of formulations. The formulations prepared at 20% theoretical loading resulted in NPs with approximately 10% loading and slow IVR (15-20% at 24 hours). If higher theoretical loading was applied the resulting NPs contained a similar amount of colistin (10%), but released colistin faster (30-40% at 24 hours).

Detailed formulations are as follows. For the decanoic acid formulation A. the oil phase was prepared with 0.246 g of decanoic acid (DA) and 1.0 g of polylactic acid-block-polyethylene glycol diblock-copolymer (PLA-PEG) (16 kDa PLA-5 kDa PEG), which were dissolved in 4 g of a mixture of ethylacetate (EA) and benzyl alcohol (BA) (79/21 w/w). Colistin sulphate solution was prepared by dissolving 0.4 g of colistin sulphate in 0.5 ml of deionized water. The water phase was prepared by dissolving 4% BA and 0.6% of Brij S100 in deionized water, and then cooling the solution to approximately 2° C. Prior to processing, 0.1 mL of NaOH solution (40% w/w) was added to the water phase. After short mixing, the solution containing 0.4 g of colistin was added to water phase. For processing, 0.1 ml of NaOH solution (40% w/w) was added to the water phase, and after short mixing, the solution containing 0.2 g of colistin was added. 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 115-120 nm in size, with a PDI of 0.15-0.18. It was found that 1 g of solid weight of nanoparticles contained 100 mg of colistin (10%).

For the decanoic acid formulation B. the oil phase was prepared with 0.123 g of decanoic acid (DA) and 1.0 g of polylactic acid-block-polyethylene glycol diblock-copolymer (PLA-PEG) (16 kDa PLA-5 kDa PEG), which were dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. The water phase was prepared by dissolving 4% BA and 0.6% of Brij S100 in deionized water, and then cooling the solution to approximately 2° C. Prior to processing, 0.1 mL of NaOH solution (40% w/w) was added to the water phase. After short mixing the solution containing 0.2 g of colistin was added to the water phase. For processing, 0.1 ml of NaOH solution (40% w/w) was added to 25 g of water phase, and after short mixing, the solution containing 0.2 g of colistin was added. 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 115-120 nm in size, with a PDI of 0.15-0.18. It was found that 1 g of solid weight of nanoparticles contained 100 mg of colistin (10%).

For the decanoic acid formulation F. the oil phase was prepared with 0.246 g of decanoic acid (DA) and 1.0 g of polylactic acid-block-polyethylene glycol diblock-copolymer (PLA-PEG) (16 kDa PLA-5 kDa PEG), which were dissolved in 4 g of a mixture of ethylacetate (EA)/benzyl alcohol (BA) (79/21 w/w). Colistin sulphate solution was prepared by dissolving 0.4 g of colistin sulphate in 0.5 ml of deionized water. The water phase was prepared by dissolving 4% BA and 0.2% of Brij 5100 in deionized water, and then cooling the solution to approximately 2° C. Prior to processing, 0.1 mL of NaOH solution (40% w/w) was added to the water phase. After short mixing the solution containing 0.4 g of colistin was added to the water phase. For processing, 5 g of oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 115-120 nm in size, with a PDI of 0.15-0.18. It was found that 1 g of solid weight of nanoparticles contained 100 mg of colistin (10%).

For the decanoic acid formulation S, the oil phase was prepared with 0.123 g of decanoic acid (DA) and 1.0 g of polylactic acid-block-polyethylene glycol diblock-copolymer (PLA-PEG) (16 kDa PLA-5 kDa PEG), which were dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. The water phase was prepared by dissolving 4% BA and 0.6% of Brij S100 in deionized water, and then cooling the solution to approximately 2° C. Prior to processing, 0.05 mL of NaOH solution (40% w/w) was added to the water phase. After short mixing, the solution containing 0.2 g of colistin was added to the water phase. For processing, 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of colistin (10%).

For the decanoic acid formulation US, the oil phase was prepared with 0.123 g of decanoic acid (DA) and 1.0 g of polylactic acid-block-polyethylene glycol diblock-copolymer (PLA-PEG) (16 kDa PLA-5 kDa PEG), which were dissolved in 4 g of a mixture of ethylacetate (EA) and benzyl alcohol (BA) (79/21 w/w). Colistin sulphate solution was prepared by dissolving 0.2 g of colistin sulphate in 0.5 ml of deionized water. The water phase was prepared by dissolving 4% BA and 0.6% of Brij S100 in deionized water, and then cooling the solution to approximately 2° C. Prior to processing, the solution containing 0.2 g of colistin was added to the water phase. For processing, 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold 0.1 M phosphate buffer at pH 7 (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of colistin (8%).

For the decanoic acid formulation I, the oil phase was prepared with 0.123 g of decanoic acid (DA) and 1.0 g of polylactic acid-block-polyethylene glycol diblock-copolymer (PLA-PEG) (10 kDa PLA-5 kDa PEG), which were dissolved in 9 g of a mixture of ethylacetate (EA) and benzyl alcohol (BA) (79/21 w/w). Colistin sulphate solution was prepared by dissolving 0.2 g of colistin sulphate in 0.5 ml of deionized water. The water phase was prepared by dissolving 4% BA and 0.4% of Brij S100 in deionized water, and then cooling the solution to approximately 2° C. Prior to processing, 0.05 mL of NaOH solution (40% w/w) was added to the water phase. After short mixing, the solution containing 0.2 g of colistin was added to the water phase. For processing, 5 g of the oil phase solution was dumped into the water phase under strong mixing. The resulting coarse emulsion was processed on a Microfluidics 110P device at 10,000 psi resulting in a fine emulsion. The fine emulsion was dumped into 600 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of colistin (10%).

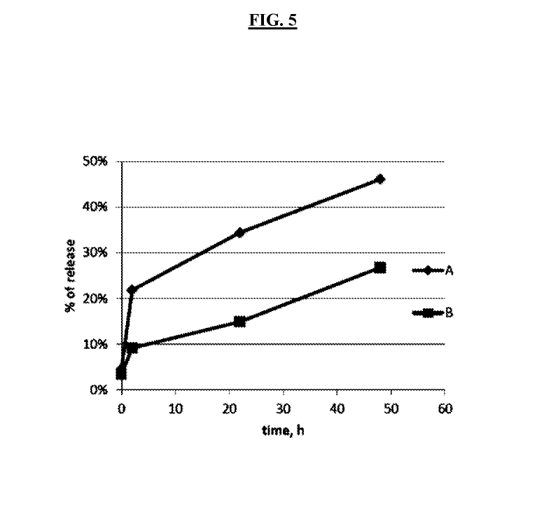

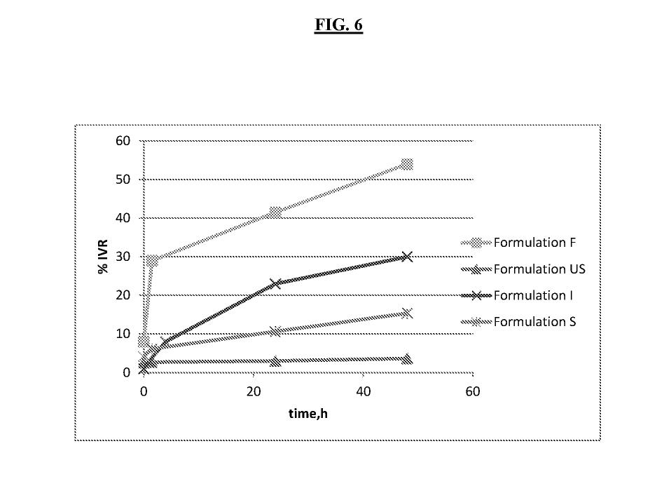

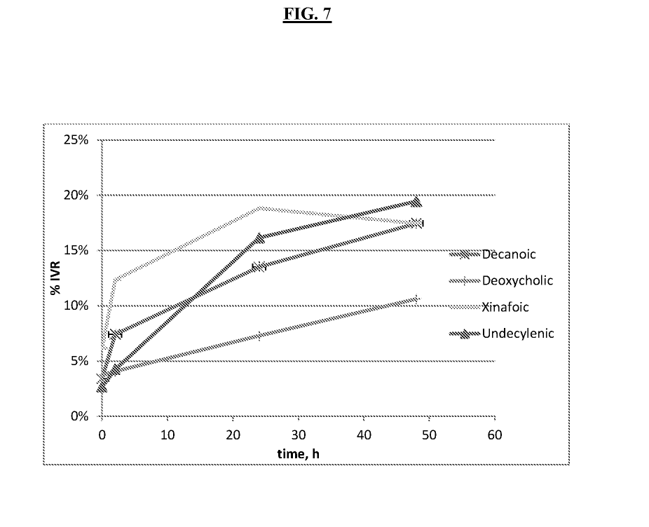

The nanoparticle in vitro release (IVR) rates are presented in FIG. 5 and FIG. 6. Formulation F and S shows similar release rates after the first hour of incubation at physiological conditions, differing in a higher release rate for formulation F during the first hour. Later data revealed that scaling up of formulation F transferred the IVR to those similar to formulation A. Moreover, conditioning of formulation F results in an IVR profile similar to formulation S. Thus, the loosely bound component of formulation B which released during first hour could be eliminated during conditioning. Nevertheless, we decided to include it in animal studies to evaluate the importance of elevated levels of the fast releasing component for biological outcomes.

In summary, colistin can be encapsulated at loads of up to 10% and colistin containing nanoparticles (CS-NPs) show sustained release of active drug for multiple days.

### Example 4: Preparation of Polymixin B-Containing Nanoparticles

Detailed formulations for Polymixin B (PMB) containing nanoparticles are as follows. For the decanoic acid formulation, the oil phase was prepared with 0.123 g of decanoic acid (DA) and 1.0 g of polylactic acid-block-polyethylene glycol diblock-copolymer (PLA-PEG) (10 kDa PLA-5 kDa PEG), which were dissolved in 4 g of a mixture of ethylacetate (EA) and benzyl alcohol (BA) (79/21 w/w). PMB sulphate solution was prepared by dissolving 0.2 g of PMB sulphate in 0.5 ml of deionized water. The water phase was prepared by dissolving 4% BA and 0.4% of Brij 5100 in deionized water, and then cooling the solution to approximately 2° C. Prior to processing, 0.05 mL of NaOH solution (40% w/w) was added to the water phase. After short mixing, the solution containing 0.2 g of PMB was added to water phase. For processing, 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and decanoic acid were removed on a tangential flow filtration system by diafilration (4 L of deionized water through a 300 kDa membrane). Resulting particles were 115-120 nm in size, with a PDI of 0.15-0.18. It was found that 1 g of solid weight of nanoparticles contained 100 mg of PMB (10%).

For the undecylenic acid formulation S, the oil phase was prepared with 0.132 g of undecylenic acid (UA) and 1.0 g of polylactic acid-block-polyethylene glycol diblock-copolymer (PLA-PEG) (10 kDa PLA-5 kDa PEG), which were dissolved in 4 g of a mixture of ethylacetate (EA) and benzyl alcohol (BA) (79/21 w/w). PMB sulphate solution was prepared by dissolving 0.2 g of PMB sulphate in 0.5 ml of deionized water. The water phase was prepared by dissolving 4% BA and 0.4% of Brij 5100 in deionized water, and then cooling the solution to approximately 2° C. Prior to processing, 0.05 mL of NaOH solution (40% w/w) was added to the water phase. After short mixing, the solution containing 0.2 g of PMB was added to the water phase. For processing, 5 g of the oil phase solution was dumped into the water phase under strong mixing. The resulting coarse emulsion was processed on a Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and undecylenic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of PMB (17%).

For the deoxycholic acid formulation, the oil phase was prepared with 1.0 g of polylactic acid-block-polyethylene glycol diblock-copolymer (PLA-PEG) (10 kDa PLA-5 kDa PEG), which was dissolved in 4 g of a mixture of ethylacetate (EA) and benzyl alcohol (BA) (79/21 w/w). PMB sulphate solution was prepared by dissolving 0.2 g of PMB sulphate in 0.5 ml of deionized water. Deoxycholic acid solution was prepared by adding 0.303 g of deoxycholic acid (DA) and 0.05 ml NaOH solution (40% w/w) to 0.45 ml of deionized water. The water phase was prepared by dissolving 4% BA and 0.4% of Brij 5100 in deionized water, and then cooling the solution to approximately 2° C. 0.5 ml of PMB solution was added to the water phase. For processing, 0.5 g of deoxycholic acid solution was added to 5 g of the oil phase solution and mixed by a high speed rotor homogenizer. The resulting emulsion was added to the water phase and mixed by a high speed rotor homogenizer. The resulting coarse emulsion was processed on a Microfluidics 110P device at 10000 psi resulting in a fine emulsion. The final emulsion was incubated for 1 minute on ice, and dumped into 300 g of cold water (<2° C.) under mixing. 28 g of Tween 80 solution in water (35% w/w) was added and mixed for 3 minutes. Downstream, the organic solvents, processing aids, non-bound drug, and deoxycholic acid were removed on a tangential flow filtration system by diafilration (4 L of deionized water through a 300 kDa membrane). Resulting particles were 100-110 nm in size, with a PDI of 0.15-0.18. It was found that 1 g of solid weight of nanoparticles contained 100 mg of PMB (10%).

For the xinafoic acid formulation, the oil phase was prepared with 0.134 g of xinafoic acid (XA) and 1.0 g of polylactic acid-block-polyethylene glycol diblock-copolymer (PLA-PEG) (10 kDa PLA-5 kDa PEG), which were dissolved in 4 g of a mixture of ethylacetate (EA) and benzyl alcohol (BA) (79/21 w/w). PMB sulphate solution was prepared by dissolving 0.2 g of PMB sulphate in 0.5 ml of deionized water. The water phase was prepared by dissolving 4% BA and 0.4% of Brij 5100 in deionized water, and then cooling the solution to approximately 2° C. Prior to processing, 0.05 mL of NaOH solution (40% w/w) was added to the water phase. After short mixing, the solution containing 0.2 g of PMB was added to water phase. For processing, 5 g of oil phase solution was dumped into the water phase under strong mixing. The resulting coarse emulsion was processed on Microfluidics 110P device at 10,000 psi resulting in a fine emulsion. The fine emulsion was dumped into 300 g of cold water (<2° C.) under mixing. 28 g of Tween 80 solution in water (35% w/w) was added and mixed for 3 minutes. Downstream, the organic solvents, processing aids, non-bound drug, and xinafoic acid were removed on a tangential flow filtration system by diafilration (4 L of deionized water through a 300 kDa membrane). Resulting particles were 115-120 nm in size, with a PDI of 0.15-0.18. It was found that 1 g of solid weight of nanoparticles contained 60 mg of PMB (10%).

Polymixin B showed similar tendencies in preparation and IVR rates relative to colistin. The particle parameters of PMB nanoparticles are summarized in Table 1, and the IVR rates are presented in FIG. 7.

TABLE 1
Counter ion selection and characterization
of Polymixin B containing nanoparticles.

| Counter ion | Loading % | size (nm) |
|---|---|---|
| Decanoic | 11 | 111.0 |
| Undecylenic | 17 | 117.2 |
| Oleic | 11 | 109.4 |
| Xinafoic | 6 | 95.5 |
| Deoxycholic | 9 | 110.8 |

### Example 5: Colistin-Containing Nanoparticles-Pharmacokinetics

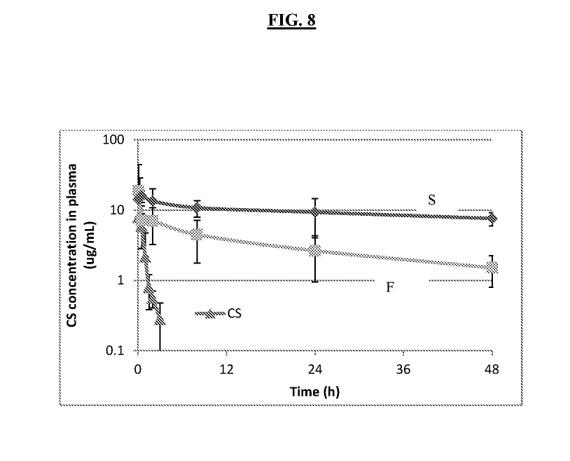

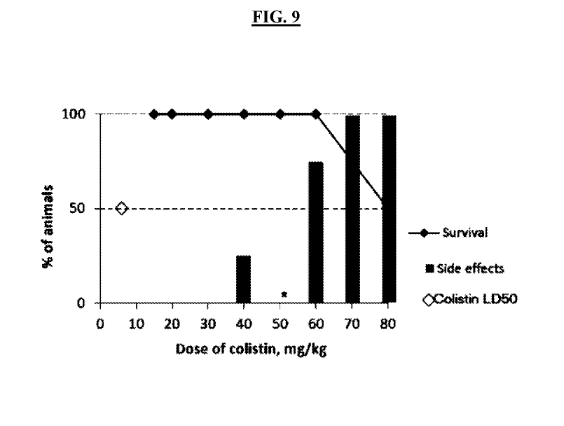

To assess the pharmacokinetics (PK) of colisitin containing nanoparticles (CS-NPs), rats were injected at a dose of 0.5 mg/kg (i.v.) with free colistin sulphate, formulation F CS-NPs, or formulation S CS-NPs. As seen in FIG. 8, CS-NPs resulted in extended circulation of colistin in the plasma, much longer than in comparison to free drug. Fast formulation F is cleared faster in comparison to slow formulation S. Moreover, the clearance from the blood stream displays similar trends as those found for IVR. Specifically, in the case of formulation F, the fast increase of released drug in IVR experiments corresponds to the initial drop of blood colisitin concentration observed in the PK experiments. For longer periods the plasma concentration decreases in nearly same rate for both formulations. CS-NP pharmacokinetics are well-differentiated from free CS and indicative of slow release of CS and retention of NPs in vascular compartments.

### Example 6: Colistin-Containing Nanoparticles-Tolerability

Tolerability was studied after intravenous injection of free drug, formulation F CS-NPs or formulation S CS-NPS. Results are presented in Table 2 and FIG. 9. Free colistin showed rather low tolerability, with an LD50 of 6 mg/kg. Mice showed a much higher LD50 and similar MTD for formulation F CS-NPs. In case of formulation S much higher tolerability was observed. The LD50 dose was over 10-fold higher in comparison to free drug (80 mg/kg), while side effects only started to appear above 40 mg/kg. Side effects of CS-NPs included lethargy and increased rate of breathing, and were observed within first minutes after dosing and resolved within 15 minutes. No BW change was observed within 14 days after dosing. Reduced toxicity of CS-NPs is likely due to retention within NPs and slow drug release, resulting in reduced systemic free colistin concentrations.

TABLE 2
Tolerability of colistin-containing nanoparticles

| Test Article | LD50 (mg/kg) | MTD (mg/kg) |
|---|---|---|
| CS | 6 | 2 |
| Formulation F CS-NPs | >10 | 6 |
| Formulation S CS-NPs | 80 | 40 |

CS-NPs were further evaluated for nephrotoxicity. Mice were dosed with 15 mg/kg of free CS (s.c.) and formulation S CS-NPs (both s.c. and i.v.) every 2 h. Mice received a total of 6 doses, and each experimental group had 5 mice. 30 hours after the first dose kidneys were collected and fixed in formalin followed by H&E staining.

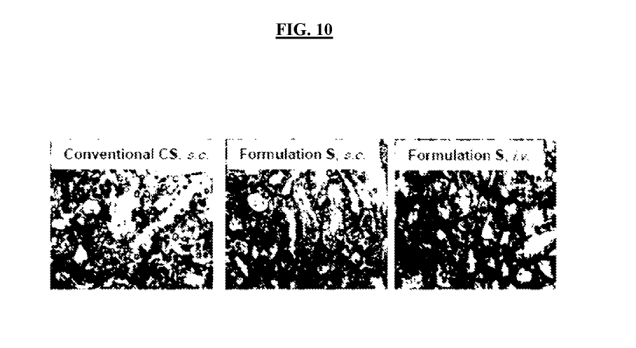

FIG. 10 shows visual changes in kidney appearance indicative of tubular degradation and necrosis in kidneys as observed by histology for each experimental group. In the free CS group 5 of 5 animals had tubular necrosis in the kidney, including multiple sites of necrosis of the tubular epithelium, and accumulation of an eosinophilic cell-free substance seen for some tubules. In both CS-NP groups (i.v. and s.c.), 5 of 5 animals did not show signs of nephrotoxicity.

In conclusion, formulation S is more tolerable and less toxic than free colistin, with a >10-fold increase in LD50, and no observed nephrotoxicity.

### Example 7: Assessing Efficacy and Biodistribution of Colistin-Containing Nanoparticles in Thigh and Lung Infection Models

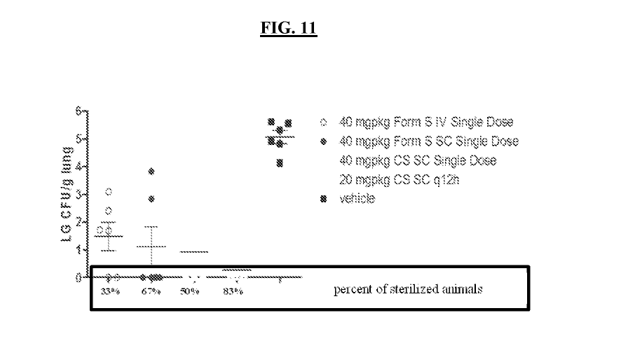

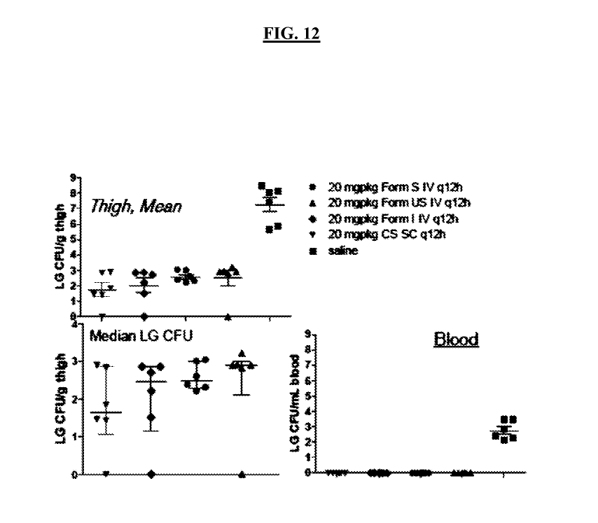

CS-NPs were tested for their efficacy in a K. pneumonia lung infection model. Results are depicted in FIG. 11. Conventional drug and formulation S CS-NPs had comparable efficacy, as 33% and 67% of animals were sterilized after s.c. and i.v. administration of a single 40 mg/kg dose of CS-NPs compared to 50% for s.c. administration of conventional drug in the same dose (n=6).

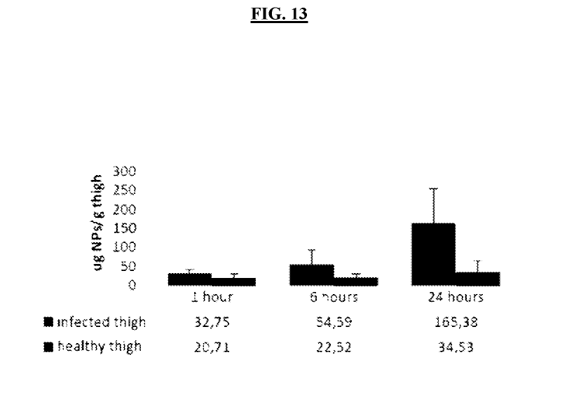

CS-NPs were further tested for their efficacy and biodistribution in a Klebsiella thigh infection model. Results are shown in FIG. 12 and FIG. 13. Treatment with both CS and CS-NPs resulted in significantly decreased bacteria counts in site of infection and prevented bacteremia, with no significant differences between the free CS and nanoparticle formulations observed after 24 hours. After 24 hours, there was a significantly higher accumulation of CS-NPs in infected compared to healthy thighs. In summary. CS-NPs have the same efficacy as free CS in a thigh infection model after 24 hours.

### Example 8: Antibacterial Activity of Antibiotic-Containing Nanoparticles In Vitro

Antibiotic loaded nanoparticles (AB-NPs) were tested for their antibacterial activity in vitro against intracellular infection as follows. 1-2×10⁶ cell/mL of THP-1 cells were activated using 0.16 μM phorbol myristate acetate. 24 hours after activation, cells were infected with F. tularensis at a bacteria-to-macrophage of 1:50. 3 hours later, THP-1 cells were washed twice to remove non-phagocytized bacteria and serial dilutions of free GNT and GNT loaded nanoparticles (GNT-NPs) were added to the cells. Cells were lysed at 3, 24 and 48 hours to determine concentration of viable bacteria (CFU) remaining in the cells. Results are shown in table

TABLE 3
Concentrations of test articles inhibiting
intracellular growth of F. tularensis.

MIC*, mg/L

| Test article | 24 hours | 48 hours |
|---|---|---|
| AB | 12-50 | 1.5-3 |
| AB-NP | 3-6 | 6-12 |

AB-NPs were more effective against intracellular F. tularensis compared to free AB at the 24 hour time point (MIC was lower than for free AB). Higher antibacterial activity of NPs at 24 hours can be explained by rapid internalization of NPs by THP-1 cells followed by AB release inside the cells, in contrast to slow penetration for the free. This in vitro study demonstrates that NPs loaded with antibiotics have a potential to treat intracellular infections.

### Example 9: Assessing the Accumulation of Vincristine-Containing Nanoparticles Versus Free Vincristine in a Mouse Thigh Infection Model

To demonstrate the potential benefits of colistin containing nanoparticles, vincristine-containing nanoparticles were tested for their ability to accumulate in infected muscle tissue in a mouse model. The levels of total vincristine in the thigh muscle of mice dosed with vincristine-containing nanoparticles was compared with those dosed with free vincristine. In addition, for each mouse, the uninfected thigh was assayed for vincristine levels to determine the ratio of vinscristine in infected versus uninfected thigh.

The study parameters were as follows: Mice:Female CD-1, immunocompetent;

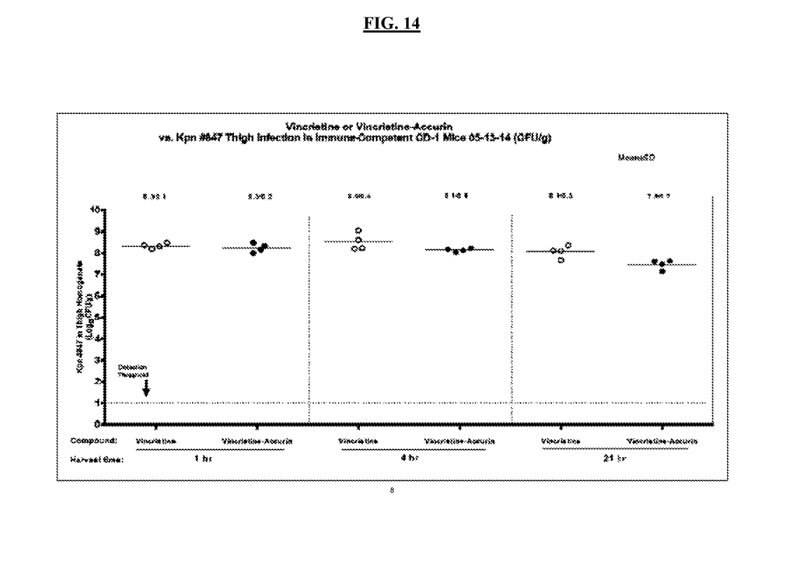

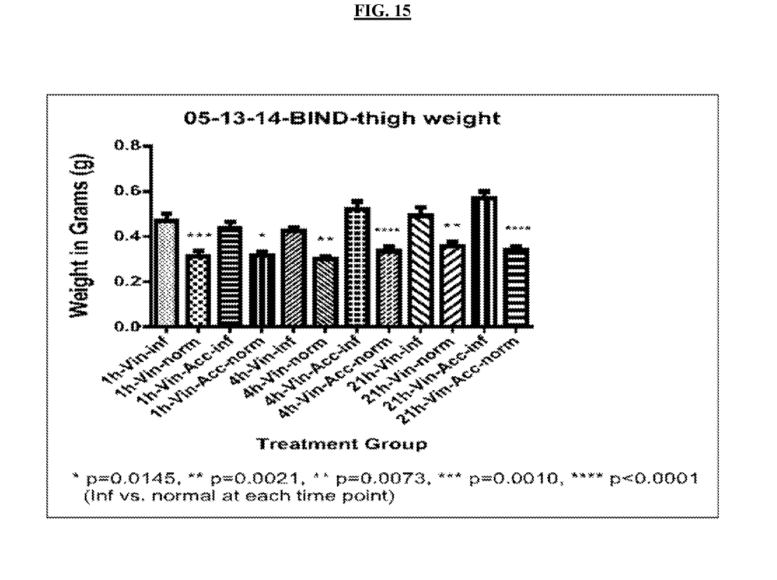

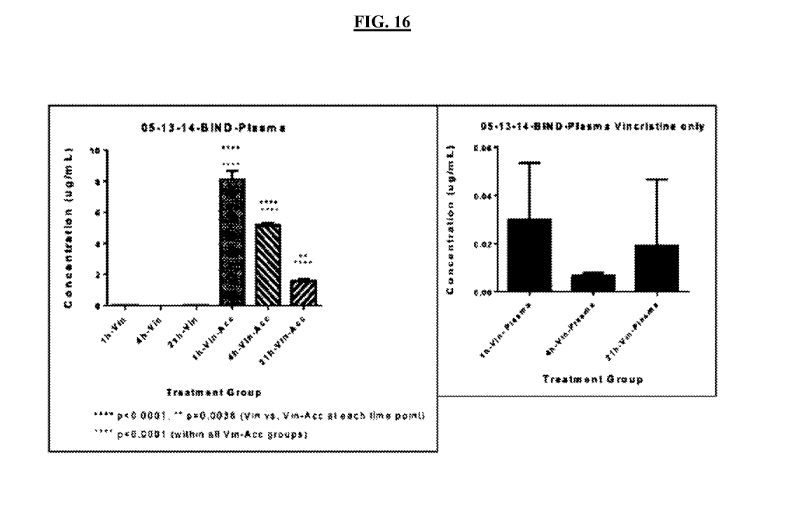

bacterium: K. pneumoniae #847; 1×108 CFU/mouse, IM; dosages, dosing regimen and route of administration of vincristine or vincristine-containing nanoparticles: 0.77 mg/kg vincristine, IV, QD (3 h post-infection); 0.77 mg/kg active API equivalent of vincristine-containing nanoparticles (total material dose of 18.6 mg/kg), IV, QD (3 h post-infection); tissue harvest time points: 1, 4 and 21 h post-dosing: left thigh (infected)-CFU determination and bioanalysis; right thigh (uninfected)-bioanalysis; blood for plasma-bioanalysis. FIG. 14 depicts a comparison of bacterial loads in mouse thighs after 1, 4, and 21 hours treated by IV with either vincristine or exemplary disclosed nanoparticle that includes vincristine. FIG. 15 depicts thigh weight of rats for infected (Left) vs. un-infected (Right). FIG. 16 depicts (Left) plasma levels of vincristine resulting from treatment with vincristine only (indicated by “Vin” axis label) vs. vincristine-containing nanoparticles (indicated by “Vin-Acc” axis label) and (Right) an expansion of the left side of the graph that shows the plasma levels of vincristine-only treated rats.

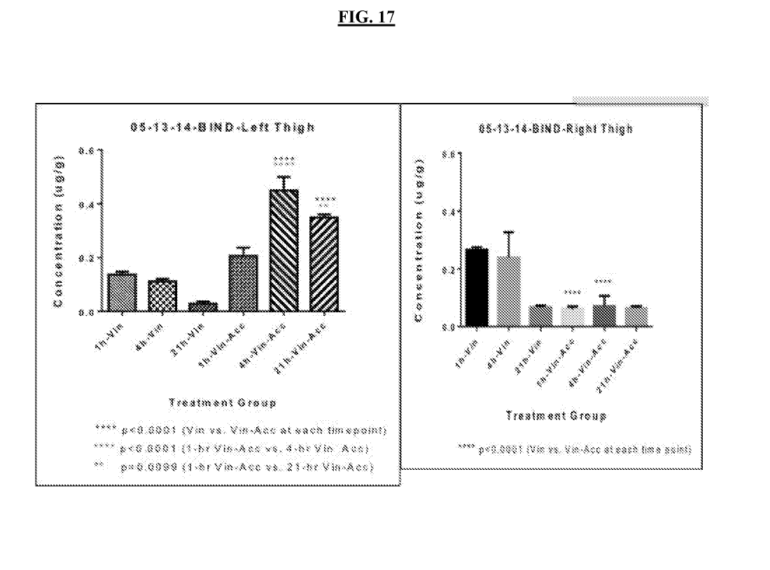

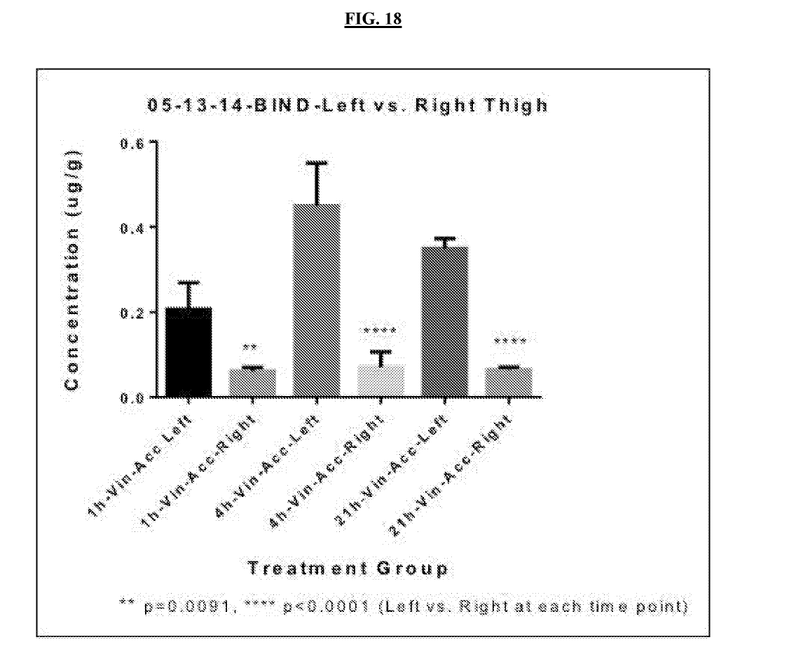

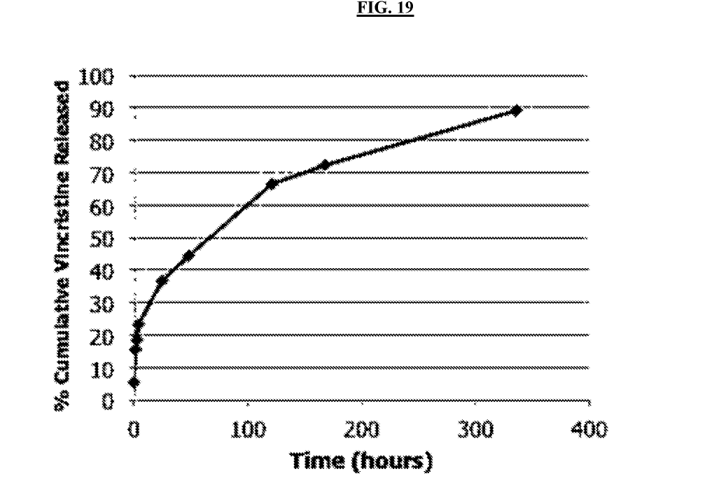

FIG. 17 depicts thigh tissue levels of vincristine vs. an exemplary disclosed nanoparticle that includes vincristine. FIG. 18 depicts thigh tissue levels of vincristine for an exemplary disclosed nanoparticle that includes vincristine for left thigh (infected) vs. right thigh (uninfected). Importantly, the therapeutic nanoparticles accumulate in infected tissue vs. uninfected tissue, thus releasing the active agent in the vicinity of the infection, thus leading to a high local concentration of active agent. FIG. 19 depicts the pharmacokinetics of vincristine release from an exemplary disclosed nanoparticle that includes vincristine in rats. As can be seen, the nanoparticles release their active agent over the course of about 350 hours.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

1. A therapeutic nanoparticle comprising:
about 0.2 to about 35 weight percent of an antibiotic therapeutic agent;
about 0.05 to about 30 weight percent of a hydrophobic acid; and
about 35 to about 99.75 weight percent of a diblock poly(lactic) acid-poly(ethylene)glycol copolymer or a diblock poly(lactic)-co-poly(glycolic) acid-poly(ethylene)glycol copolymer, wherein the therapeutic nanoparticle comprises about 10 to about 30 weight percent poly(ethylene)glycol.

2. The therapeutic nanoparticle of claim 1, wherein the antibiotic is polymyxin B.

3. The therapeutic nanoparticle of claim 1, wherein the antibiotic is polymyxin E.

4. The therapeutic nanoparticle of claim 1, wherein the hydrophobic acid is selected from deanoic acid (DEC), pamoic acid (PAM), xinaphoic acid (XIN), and deocycholic acid (DEOC).

5. The therapeutic nanoparticle of claim 1, wherein the hydrophobic acid is a bile acid or a fatty acid.

6. The therapeutic nanoparticle of claim 5, wherein the fatty acid is a saturated fatty acid selected from the group consisting of: caproic acid, enanthic acid, caprylic acid, pelargonic acid, capric acid, undecanoic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, and combinations thereof.

7. The therapeutic nanoparticle of claim 5 wherein the fatty acid is an omega-3 fatty acid selected from the group consisting of: hexadecatrienoic acid, alpha-linolenic acid, stearidonic acid, eicosatrienoic acid, eicosatetraenoic acid, eicosapentaenoic acid, heneicosapentaenoic acid, docosapentaenoic acid, docosahexaenoic acid, tetracosapentaenoic acid, tetracosahexaenoic acid, and combinations thereof.

8. The therapeutic nanoparticle of claim 5, wherein the fatty acid is an omega-6 fatty acid selected from the group consisting of: linoleic acid, gamma-linolenic acid, eicosadienoic acid, dihomo-gamma-linolenic acid, arachidonic acid, docosadienoic acid, adrenic acid, docosapentaenoic acid, tetracosatetraenoic acid, tetracosapentaenoic acid, and combinations thereof.

9. The therapeutic nanoparticle of claim 5, wherein the fatty acid is an omega-9 fatty acid selected from the group consisting of: oleic acid, eicosenoic acid, mead acid, erucic acid, nervonic acid, and combinations thereof.

10. The therapeutic nanoparticle of claim 5, wherein the fatty acid is a polyunsaturated fatty acid selected from the group consisting of: rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, podocarpic acid, and combinations thereof.

11. The therapeutic nanoparticle of claim 1, wherein the hydrophobic acid is selected from the group consisting of 1-hydroxy-2-naphthoic acid, dodecylsulfuric acid, naphthalene-1,5-disulfonic acid, naphthalene-2-sulfonic acid, pamoic acid, undecanoic acid, and combinations thereof.

12. The therapeutic nanoparticle of claim 1, wherein the bile acid is selected from the group consisting of chenodeoxycholic acid, ursodeoxycholic acid, deoxycholic acid, hycholic acid, beta-muricholic acid, cholic acid, an amino acid-conjugated bile acid, and combinations thereof.

13. The therapeutic nanoparticle of claim 12, wherein the amino acid-conjugated bile acid is a glycine-conjugated bile acid or a taurine-conjugated bile acid.

14. The therapeutic nanoparticle of claim 1, wherein the hydrodynamic diameter of the therapeutic nanoparticle is about 60 to about 150 nm.

15. The therapeutic nanoparticle of claim 1, wherein the hydrodynamic diameter is about 90 to about 140 nm.

16. The therapeutic nanoparticle of claim 1, comprising about 1 to about 15 weight percent of the antibiotic therapeutic agent.

17. The therapeutic nanoparticle of claim 1, comprising about 4 to about 15 weight percent of the antibiotic therapeutic agent.

18. The therapeutic nanoparticle of claim 1, comprising about 5 to about 10 weight percent of antibiotic therapeutic agent.

19. The therapeutic nanoparticle of claim 1, comprising about 2 to about 5 weight percent of the antibiotic therapeutic agent.

20. The therapeutic nanoparticle of claim 1, wherein the therapeutic nanoparticle substantially retains the antibiotic therapeutic agent for at least 1 minute when placed in a phosphate buffer solution at 37° C.

21. The therapeutic nanoparticle of claim 1, wherein the therapeutic nanoparticle substantially immediately releases less than about 30% of the antibiotic therapeutic agent when placed in a phosphate buffer solution at 37° C.

22. The therapeutic nanoparticle of claim 1, further comprising about 0.2 to about 30 weight percent poly(lactic) acid-poly(ethylene)glycol copolymer functionalized with a targeting ligand.

23. The therapeutic nanoparticle of claim 1, wherein the polylactic portion of the diblock polymer has a number average molecule weight of about 15 kDa to about 18 kda, and the poly(ethylene)glycol portion has a number average molecular weight of about 4 kDa to about 6 kDa.

24. A pharmaceutically acceptable composition comprising a plurality of therapeutic nanoparticles of claim 1 and a pharmaceutically acceptable excipient.

25. A method of treating inflammation and/or infection in a patient in need thereof, comprising administering to the patient a therapeutically effective amount of a composition comprising the therapeutic nanoparticle of claim 1.


SEQUENCE LISTING (4 sequences)

SEQ ID NO: 1 - protein, 5 aa, Artificial Sequence
  Synthetic peptide
AKERC

SEQ ID NO: 2 - protein, 5 aa, Artificial Sequence
  Synthetic peptide
CREKA

SEQ ID NO: 3 - protein, 8 aa, Artificial Sequence
  Synthetic peptide
ARYLQKLN

SEQ ID NO: 4 - protein, 8 aa, Artificial Sequence
  Synthetic peptide
  misc_feature (2)..(2): Xaa can be any naturally occurring amino acid
  misc_feature (5)..(6): Xaa can be any naturally occurring amino acid
AXYLXXLN